SEQUENCE LISTING (309 sequences)

SEQ ID NO: 1 - protein, 56 aa, Human immunodeficiency virus
LLQLTVWGIKQLQARVLAVERYLRDQQLLGIWGCSGKLICTTTVPWNTSWSNKSLD

SEQ ID NO: 2 - protein, 56 aa, Human immunodeficiency virus
LQLTVWGIKQLRARVLAVERYLRDQQLLGIWGCSGKLICTTNVPWNSSWSNKSQSE

SEQ ID NO: 3 - protein, 56 aa, Human immunodeficiency virus
AQQHLLQLTVWGIKQLQARVLAVERYLRDQQLLGIWGCSGKLICTTTVPWNASWSN

SEQ ID NO: 4 - protein, 56 aa, Human immunodeficiency virus
KSIRIGPGQAFYATGDIIGDIRRAYCNISGKQWNETLHKVITKLGSYFDNKTIILQ

SEQ ID NO: 5 - protein, 56 aa, Human immunodeficiency virus
QQHLLQLTVWGIKQLQARVLAVERYLKDQQLLGIWGCSGKLICTTTVPWNASWSNK

SEQ ID NO: 6 - protein, 56 aa, Human immunodeficiency virus
IWGCSGKLICPTTVPWNLSWSNKSQDEIWGNMTWMEWEKEIGNYTDTIYRLIESAQ

SEQ ID NO: 7 - protein, 56 aa, Human immunodeficiency virus
QARILAVERYLKDQQLLGFWGCSGKLICTTAVPWNASWSNKTLDQIWNNMTWMEWD

SEQ ID NO: 8 - protein, 56 aa, Human immunodeficiency virus
LQDQRLLGMWGCSGKHICTTFVPWNSSWSNRSLDDIWNNMTWMQWEKEISNYTGII

SEQ ID NO: 9 - protein, 56 aa, Human immunodeficiency virus
WGIKQLQARVLAVERYLRDQQLLGIWGCSGRHICTTNVPWNSSWSNRSLDEIWQNM

SEQ ID NO: 10 - protein, 56 aa, Human immunodeficiency virus
QLTVWGIKQLQARILAVERYLKDQQLLGIWGCSGKLICPTTVPWNLSWSNKSQDEI

SEQ ID NO: 11 - protein, 56 aa, Human immunodeficiency virus
IIVQLNESVEITCTRPSNNTRESIRIGPGQTFYATGDIIGDIRQAHCNISEEKWNK

SEQ ID NO: 12 - protein, 56 aa, Human immunodeficiency virus
ESVQINCTRPNNNTRKGIHLGPGQTFYATGAIIGDIRKAHCNISGTQWNNTLEYVK

SEQ ID NO: 13 - protein, 56 aa, Human immunodeficiency virus
QQLLGIWGCSGKLICTTTVPWNASWSNKSLNEIWDNMTWMQWEREIDNYTHLIYTL

SEQ ID NO: 14 - protein, 56 aa, Human immunodeficiency virus
AQQHLLKLTVWGIKQLQARILAVERYLKDQQLLGIWGCSGKIICPTNVPWNSSWSN

SEQ ID NO: 15 - protein, 56 aa, Human immunodeficiency virus
MLELTVWGIKQLQARVLAVERYLKDQQLLGIWGCSGKLICTTAVPWNASWSNKSLS

SEQ ID NO: 16 - protein, 56 aa, Human immunodeficiency virus
KQLQARVLAVERYLRDQQLLGIWGCSGKLICTTTVPWNTSWSNKSLNEIWDNMTWM

SEQ ID NO: 17 - protein, 56 aa, Human immunodeficiency virus
IGPGQTFFATGEIIGDIRKAYCNINRTLWNETLKNVSGEFKKHFNFSVAFNSSSGG

SEQ ID NO: 18 - protein, 56 aa, Human immunodeficiency virus
QLQSRVLAIERYLKDQQLLGIWGCSGKLICTTNVPWNTSWSNKSYNEIWDNMTWLE

SEQ ID NO: 19 - protein, 56 aa, Human immunodeficiency virus
LLGIWGCSGKHICTTNVPWNSSWSNRSLNEIWQNMTWMEWEREIDNYTGLIYSLIE

SEQ ID NO: 20 - protein, 56 aa, Human immunodeficiency virus
QKQITKIQNFRVYYRDSRNPLWKGPAKLLWKGEGAVVIQDNSDIKVVPRRKAKIIR

SEQ ID NO: 21 - protein, 56 aa, Human immunodeficiency virus
KQLQARILAVERYLKDQQLLGIWGCSGKLICTTNVRWNSSWSNKSYDDIWDNMTWM

SEQ ID NO: 22 - protein, 56 aa, Human immunodeficiency virus
NGSLAEEGIVIRSQNISNNAKTIIVHLNESVQINCTRPNNNTRKGIHLGPGQTFYA

SEQ ID NO: 23 - protein, 56 aa, Human immunodeficiency virus
LTVWGIKQLQTRVLAIERYLRDQQLLGIWGCSGKLICTTAVPWNSSWSNRSQEDIW

SEQ ID NO: 24 - protein, 56 aa, Human immunodeficiency virus
NNMTWMQWDREISNYTNTIYRLLEDSQNQQEKNEQDLLALDKWQNLWTWFGITNWL

SEQ ID NO: 25 - protein, 56 aa, Human immunodeficiency virus
ARILAVERYLKDQQLLGIWGCSGKLICTTAVPWNASWSNKSLEEIWDNMTWMEWER

SEQ ID NO: 26 - protein, 56 aa, Human immunodeficiency virus
QNFRVYYRDNRDPIWKGPAKLLWKGEGAVVIQDNSDIKVVPRRKAKIIRDYGKQMA

SEQ ID NO: 27 - protein, 56 aa, Human immunodeficiency virus
CSGKLICTTAVPWNASWSNKSLDQIWNNMTWMEWEREIDNYTNLIYTLIEESQNQQ

SEQ ID NO: 28 - protein, 56 aa, Human immunodeficiency virus
WGIKQLQARVLAVERYLKDQQLLGLWGCSGKLICTTNVPWNSSWSNKSLEEIWGNM

SEQ ID NO: 29 - protein, 56 aa, Human immunodeficiency virus
VIIVQLNNSIEINCIRPNNNTRKSIPIGPGQAFYATGDIIGDIRQAHCNVSRIKWR

SEQ ID NO: 30 - protein, 56 aa, Human immunodeficiency virus
YMLKHLVWANRELEKFALNPDLLDTSAGCKQIIKQLQPALQTGTEELKSLFNTVAT

SEQ ID NO: 31 - protein, 56 aa, Human immunodeficiency virus
NFRVYYRDSRDPIWKGPAKLLWKGEGAVVIQDNSDIKVVPRRKAKIIRDYGKQMAG

SEQ ID NO: 32 - protein, 56 aa, Human immunodeficiency virus
GIKQLQARVLAVERYLRDQQLLGIWGCSGKLICTTNVPWNSSWSNKSQSEIWDNMT

SEQ ID NO: 33 - protein, 56 aa, Human immunodeficiency virus
AIQAQQHMLQLTVWGIKQLQARVLAVERYLKDQQLLGIWGCSGKLICTTNVPWNSS

SEQ ID NO: 34 - protein, 56 aa, Human immunodeficiency virus
RARLQALETLIQNQQRLNLWGCKGKLICYTSVKWNTSWSGRYNDDSIWDNLTWQQW

SEQ ID NO: 35 - protein, 56 aa, Human immunodeficiency virus
GIWGCSGKLICTTNVPWNSSWSNKSQSEIWENMTWMQWEKEISNHTSTIYRLIEES

SEQ ID NO: 36 - protein, 56 aa, Human immunodeficiency virus
DQQLLGIWGCSGKIICPTNVPWNSSWSNKSQSDIWDKMTWLEWDKEVSNYTQVIYN

SEQ ID NO: 37 - protein, 56 aa, Human immunodeficiency virus
ITDNTKNIIVQLNEPVQINCTRTGNNTRKSIRIGPGQAFYATGDIIGDIRRAYCNI

SEQ ID NO: 38 - protein, 56 aa, Human immunodeficiency virus
LLRAIDAQQHLLQLTVWGIKQLQARVLAVERYLKDQQLMGIWGCSGKFICTTAVPW

SEQ ID NO: 39 - protein, 56 aa, Human immunodeficiency virus
NTKTIIVQFKNPVKINCTRPNNNTRRSIHIGPGRAFYATGEIIGDTRKAHCNISEK

SEQ ID NO: 40 - protein, 56 aa, Human immunodeficiency virus
LRAIEAQQHLLQLTVWGIKQLQARILAVERYLKDQQLLGIWGCSGKLICTTAVPWN

SEQ ID NO: 41 - protein, 56 aa, Human immunodeficiency virus
LMGIWGCSGKLICTTSVPWNVSWSNKSVDDIWNNMTWMEWEREIDNYTDYIYDLLE

SEQ ID NO: 42 - protein, 56 aa, Human immunodeficiency virus
TRPSKTTRRRIHIGPGRAFYTTKQIAGDLRQAHCNINRARWNATLKQIVGKLRKQF

SEQ ID NO: 43 - protein, 56 aa, Human immunodeficiency virus
AVESYLKDQQLLGIWGCSGKHICTTTVPWNSSWSNKSLEEIWNNMTWIEWEREIDN

SEQ ID NO: 44 - protein, 56 aa, Human immunodeficiency virus
ATLEEMMTACQGVGGPGHKARVLAEAMSQVQQTSIMMQRGNFRGPRRIKCFNCGKE

SEQ ID NO: 45 - protein, 56 aa, Human immunodeficiency virus
IISTDIQTRELQKQITKIQNFRVYYRDSRNPVWKGPAKLLWKGEGAVVIQDNSEIK

SEQ ID NO: 46 - protein, 56 aa, Human immunodeficiency virus
WSNKSLDEIWDNMTWMEWDKQINNYTDEIYRLLEVSQNQQEKNEQDLLALDKWANL

SEQ ID NO: 47 - protein, 56 aa, Human immunodeficiency virus
VERYLKDQQLMGIWGCSGKFICTTAVPWNTSWSNKSFNEIWDNMTWMEWEREINNY

SEQ ID NO: 48 - protein, 56 aa, Human immunodeficiency virus
IKQLQTRVLAIERHLRDQQLLGIWGCSGKLICTTAVPWNSSWSNKSQEEIWDNMTW

SEQ ID NO: 49 - protein, 56 aa, Human immunodeficiency virus
YKLKHIVWASRELERFAVNPGLLETSEGCRQILGQLQPSLQTGSEELRSLYNTVAT

SEQ ID NO: 50 - protein, 56 aa, Human immunodeficiency virus
GCSGKLICTTAVPWNTSWSNKSLDSIWNNMTWMEWEKEIENYTNTIYTLIEESQIQ

SEQ ID NO: 51 - protein, 56 aa, Human immunodeficiency virus
QQHLLQLTVWGIKQLQARILAVERYLKDQQLLGIWGCSGKLICTTTVPWNSSWSNR

SEQ ID NO: 52 - protein, 56 aa, Human immunodeficiency virus
QQQNNLLRAIEGQQHLLQLTVWGIKQLQARILAVERYLKDQQLLGIWGCSGKLICT

SEQ ID NO: 53 - protein, 56 aa, Human immunodeficiency virus
YRLKHIVWASRKLERFAVNPGLLETSKGCRQILGQLQPSLQTGSEELRSLYNTVAT

SEQ ID NO: 54 - protein, 56 aa, Human immunodeficiency virus
GIVQQQSNLLRAIEAQQHLLQLTVWGIKQLRARILAVERYLKDQQLLGIWGCSGKL

SEQ ID NO: 55 - protein, 56 aa, Human immunodeficiency virus
SWSNKSQEEIWNNMTWMEWEKEISNYSNIIYKLIEESQNQQEKNEQELLALDKWAS

SEQ ID NO: 56 - protein, 56 aa, Human immunodeficiency virus
YRLKHLVWASRELERFALDPGLLETSEGCRKIIGQLQPSLQTGSEELKSLYNTIAV

SEQ ID NO: 57 - protein, 56 aa, Human immunodeficiency virus
LGIWGCSGKLICTTAVPWNASWSNKSLSDIWDNMTWMEWEREIDNYTNLIYSLIED

SEQ ID NO: 58 - protein, 56 aa, Human immunodeficiency virus
YRLKHIVWASRELERYALNPGLLETSEGCKQIIGQLQPAIQTGTEELRSLYNTVAT

SEQ ID NO: 59 - protein, 56 aa, Human immunodeficiency virus
QQHLLQLTVWGIKQLQARVLAVERYLRDQQLLGIWGCSGKLICTTAVPWNASWSNK

SEQ ID NO: 60 - protein, 56 aa, Human immunodeficiency virus
GHIARNCRAPRKKGCWKCGREGHQMKDCTERQANFFRENLAFQQGEARKFPSEQTR

SEQ ID NO: 61 - protein, 56 aa, Human immunodeficiency virus
WGCSGKLICTTAVPWNSSWSNRSQEDIWNNMTWMQWDREISNYTNTIYRLLEDSQN

SEQ ID NO: 62 - protein, 56 aa, Human immunodeficiency virus
IIVQLNETVKINCTRPGSDKKIRQSIRIGPGKVFYAKGGITGQAHCNITDGEWRNT

SEQ ID NO: 63 - protein, 56 aa, Human immunodeficiency virus
SGIVQQQSNLLKAIEAQQHLLQLTVWGIKQLQARILAVERYLKDQQLLGIWGCSGK

SEQ ID NO: 64 - protein, 56 aa, Human immunodeficiency virus
TIAYIEYRKLLRQRRIDRLIKRTRERAEDSGNESDGDTEELSTMVDMGNLRLLDVN

SEQ ID NO: 65 - protein, 56 aa, Human immunodeficiency virus
TKNIIVQFNRSVIIDCRRPNNNTRKGIRIGPGQTFFATGEIIGDIRKAYCNINRTL

SEQ ID NO: 66 - protein, 56 aa, Human immunodeficiency virus
TIIVHLNESVEINCTRPNNNTRKSIRIGPGQAFYATGEIIGDIRQAHCNISRTAWN

SEQ ID NO: 67 - protein, 56 aa, Human immunodeficiency virus
ERYLKDQQLLGIWGCSGKLICTTAVPWNASWSNKSLEQIWNNMTWMEWDREINNYT

SEQ ID NO: 68 - protein, 56 aa, Human immunodeficiency virus
YMMKHLVWASRELERFALDPGLLETSEGCKQIMKQLQPALQTGTKELISLHNTVAT

SEQ ID NO: 69 - protein, 56 aa, Human immunodeficiency virus
AQLNSWGCAFRQVCHTTVPWVNDSLSPDWKNMTWQEWEKQVRYLEANISQSLEEAQ

SEQ ID NO: 70 - protein, 56 aa, Human immunodeficiency virus
AKTIIVHLNESVEINCTRPNNNVRRRHIHIGPGRAFYTGEIRGNIRQAHCNISRAK

SEQ ID NO: 71 - protein, 56 aa, Human immunodeficiency virus
YKMKHLIWASRELERFALDPGLLETSEGCQKIIRQLQPSLQTGSEELKSLFNTVAV

SEQ ID NO: 72 - protein, 56 aa, Human immunodeficiency virus
DNAKTIIVHLNESVQINCTRPNNNTRKRISLGPGRVFYTTGEIIGDIRKAHCNVSG

SEQ ID NO: 73 - protein, 56 aa, Human immunodeficiency virus
RPNNHTRKRVTLGPGRVWYTTGEILGNIRQAHCNISRAQWNNTLQQIATTLREQFG

SEQ ID NO: 74 - protein, 56 aa, Human immunodeficiency virus
VYYRDSRDPIWKGPAQLLWKGEGAVVIQDKGDIKVVPRRKAKIIRDYGKQMAGTDS

SEQ ID NO: 75 - protein, 56 aa, Human immunodeficiency virus
NNTRKSIHIGPGRAFYATGDIIGDIRQAHCNISGGQWNKTVNQVKKELGKHFNKTI

SEQ ID NO: 76 - protein, 56 aa, Human immunodeficiency virus
KLICTTAVPWNSSWSNKSQEEIWDNMTWMQWDREISNYTDIIYNLLEVSQNQQDKN

SEQ ID NO: 77 - protein, 56 aa, Human immunodeficiency virus
YLKDQQLLGIWGCSGKLICTTNVPWNSSWSNKSLDEIWDNMTWMEWDKQINNYTDE

SEQ ID NO: 78 - protein, 56 aa, Human immunodeficiency virus
ATLEEMMTACQGVGGPSHKARVLAEAMSQANSAIMMQKSNFKGQRRVVKCFNCGKE

SEQ ID NO: 79 - protein, 56 aa, Human immunodeficiency virus
TVWGIKQLQARVLAVERYLKDQQLMGIWGCSGKLICTTAVPWNTSWSNKSLDSIWN

SEQ ID NO: 80 - protein, 56 aa, Human immunodeficiency virus
RAIEAQQHLLQLTVWGIKQLQARVLAVERYLKDQQLLGIWGCSGKLICTTNVPWNS

SEQ ID NO: 81 - protein, 56 aa, Human immunodeficiency virus
DNTKNIIVQLNETVQINCTRPNNNTRKSIHMGPGKAFYTTGDIIGDIRQAHCNISG

SEQ ID NO: 82 - protein, 56 aa, Human immunodeficiency virus
YYRDSRDPIWKGPAKLLWKGEGAVVIQDNNEIKVVPRRKAKILKDYGKQMAGGDCV

SEQ ID NO: 83 - protein, 56 aa, Human immunodeficiency virus
SENFTDNVKTIIVQLNETVKINCIRPNNKTRKRVTMGPGRVYYTTGEIIGDIRQAH

SEQ ID NO: 84 - protein, 56 aa, Human immunodeficiency virus
EVVIRSENFTDNAKTIIVQLKEPVVINCTRPSKTTRRRIHIGPGRAFYTTKQIAGD

SEQ ID NO: 85 - protein, 56 aa, Human immunodeficiency virus
LKTPVNITCTRPNNNTRTSIHLGPGRAFYATGDIIGDIRQAHCNISRTDWNKTLHQ

SEQ ID NO: 86 - protein, 56 aa, Human immunodeficiency virus
EAQQHMLQLTVWGIKQLQARVLAVERYLKDQQLLGFWGCSGKLICTTTVPWNASWS

SEQ ID NO: 87 - protein, 56 aa, Human immunodeficiency virus
HLLQLTVWGIKQLQARILAVERYLKDQQLLGIWGCSGKHICTTNVPWNSSWSNRSL

SEQ ID NO: 88 - protein, 56 aa, Human immunodeficiency virus
YRLKHLVWASRELERFALNPGLLETSDGCKQIIGQLQPAIRTGSEELRSLFNTVAT

SEQ ID NO: 89 - protein, 56 aa, Human immunodeficiency virus
YRLKHIVWASRELKRFALNPGLLETTEGCKKIIGQLQPSLQTGSEELKSLFNTIVV

SEQ ID NO: 90 - protein, 56 aa, Human immunodeficiency virus
RYLKDQQLLGIWGCSGKLICTTNVPWNSSWSNKSQEEIWNNMTWMEWEKEISNYSN

SEQ ID NO: 91 - protein, 56 aa, Human immunodeficiency virus
SLEDIWDNMTWMQWEREIDNYTNTIYTLLEESQNQQEKNEQELLELDKWASLWNWF

SEQ ID NO: 92 - protein, 56 aa, Human immunodeficiency virus
YRIKHLVWASRELDRFALNPGLLESAKGCQQILVQLQPALQTGTQEIKSLYNTVAT

SEQ ID NO: 93 - protein, 56 aa, Human immunodeficiency virus
QLRARILAVERYLKDQQLLGIWGCSGKLICTTNVPWNSSWSNKSWEEIWNNMTWME

SEQ ID NO: 94 - protein, 56 aa, Human immunodeficiency virus
DQQLLGIWGCSGKLICTTTVPWNASWSNKSMDQIWNNMTWMEWEREIDNYTSLIYN

SEQ ID NO: 95 - protein, 56 aa, Human immunodeficiency virus
VWGIKQLQARVLAVERYLKDQQLLGIWGCSGKLICTTAVPWNASWSNKSLDQIWNN

SEQ ID NO: 96 - protein, 56 aa, Human immunodeficiency virus
LKDQALLNSWGCAFRQVCHTTVPWPNETLTPDWENMTWQQWEKRVNFLDANITALL

SEQ ID NO: 97 - protein, 56 aa, Human immunodeficiency virus
YSAGERIIDIIASDIQTKELQNQILKIQNFRVYYRDSRDPIWKGPAKLLWKGEGAV

SEQ ID NO: 98 - protein, 56 aa, Human immunodeficiency virus
RKRIHIGPGRAFYTTKNIIGTIRQAHCNISRAKWNDTLRQIVSKLKEQFKNKTIVF

SEQ ID NO: 99 - protein, 56 aa, Human immunodeficiency virus
TLTVQARQLMSGIVQQQNNLLRAIEAQQHLLQLTVWGIKQLQARILAVERYLKDQQ

SEQ ID NO: 100 - protein, 56 aa, Human immunodeficiency virus
YKLKHIVWASRELGRFALNRDLLETAEGCVQIMKQLQPALTGTEELRSLFNTVATL

SEQ ID NO: 101 - protein, 56 aa, Human immunodeficiency virus
QQLLGIWGCSGKLICTTAVPWNASWSNKSLEDIWDNMTWMQWEREIDNYTNTIYTL

SEQ ID NO: 102 - protein, 56 aa, Human immunodeficiency virus
KTRKRVTMGPGRVYYTTGEIIGDIRQAHCNISRAEWNKTLEQIANKLRKQFENKTI

SEQ ID NO: 103 - protein, 56 aa, Human immunodeficiency virus
WGNMTWMEWEKEIGNYTDTIYRLIESAQNQQEKNEQDLLALDKWDNLWNWFSITRW

SEQ ID NO: 104 - protein, 56 aa, Human immunodeficiency virus
RSEDITKNTKNIIVQLNEAVEINCTRPSNNTRKSIHIGPGRAFYATGDIIGDIRQA

SEQ ID NO: 105 - protein, 56 aa, Human immunodeficiency virus
DSWTVNDIQKLVGKLNWASQIYPGIKVRQLCKLLRGAKALTDIVTLTEEAELELAE

SEQ ID NO: 106 - protein, 56 aa, Human immunodeficiency virus
TIIVQLKESVEINCTRPNNNTRKSITIGPGRAFYATGDIIGDIRQAHCNISGEKWN

SEQ ID NO: 107 - protein, 56 aa, Human immunodeficiency virus
NKSLDDIWNNMTWMQWEREIDNYTSLIYSLLEKSQTQQEKNEQELLELDKWASLWN

SEQ ID NO: 108 - protein, 56 aa, Human immunodeficiency virus
TIAYIEYRKLVRQRRIDWLVKRIKERAEDSGNESGGDTEELETMVDMGHLRLLDGN

SEQ ID NO: 109 - protein, 56 aa, Human immunodeficiency virus; identical to SEQ ID NO: 30

SEQ ID NO: 110 - protein, 56 aa, Human immunodeficiency virus
QARLNSWGCAFRQVCHTTVPWVNDSLAPDWDNMTWQEWEKQVRYLEANISKSLEQA

SEQ ID NO: 111 - protein, 56 aa, Human immunodeficiency virus
ICTTNVPWNTSWSNKSYNEIWDNMTWLEWEREIHNYTQHIYSLIEESQNQQEKNEQ

SEQ ID NO: 112 - protein, 56 aa, Human immunodeficiency virus
HMLQLTVWGIKQLQARVLALERYLRDQQLMGIWGCSGKLICTTSVPWNVSWSNKSV

SEQ ID NO: 113 - protein, 56 aa, Human immunodeficiency virus
IEAQQHLLQLTVWGIKQLQARVLAVERYLQDQRLLGMWGCSGKHICTTFVPWNSSW

SEQ ID NO: 114 - protein, 56 aa, Human immunodeficiency virus
YKLKHLVWASRELERFALNPGLLETTEGCRQIITQIQPSIQTGSEEIKSLYNTIAV

SEQ ID NO: 115 - protein, 56 aa, Human immunodeficiency virus
ALGPQATLEEMMTACQGVGGPSHKARVLAEAMSQATNSAAAVMMQRGNFKGPRKTI

SEQ ID NO: 116 - protein, 56 aa, Human immunodeficiency virus
TNNAKTIIVQLNESVAINCTRPNNNTRKSIYIGPGRAFHTTGRIIGDIRKAHCNIS

SEQ ID NO: 117 - protein, 56 aa, Human immunodeficiency virus
KDQAQLNSWGCAFRQVCHTTVPWPNETLTPNWNNMTWQQWEKQVHFLDANITALLE

SEQ ID NO: 118 - protein, 56 aa, Human immunodeficiency virus
YRLKHLVWASRELERFALNPGLLETGEGCQQIMEQLQSTLKTGSEEIKSLYNTVAT

SEQ ID NO: 119 - protein, 56 aa, Human immunodeficiency virus
VLAVERYLRDQQLLGIWGCSGKLICTTTVPWNASWSNKSLNMIWNNMTWMQWEREI

SEQ ID NO: 120 - protein, 56 aa, Human immunodeficiency virus
ELREHLLRWGFTTPDKKHQKEPPFLWMGHELHPDKWTVQPIQLPNKDSWTVNDIQK

SEQ ID NO: 121 - protein, 56 aa, Human immunodeficiency virus
VQQQNNLLRAIKAQQHLLQLTVWGIKQLQARILAVERYLKDQQLLGFWGCSGKLIC

SEQ ID NO: 122 - protein, 56 aa, Human immunodeficiency virus
QLNSWGCAFRQVCHTTVPWVNESLKPDWNNMTWQQWERQVRFLDANITKLLEEAQI

SEQ ID NO: 123 - protein, 56 aa, Human immunodeficiency virus
NLLRAIEAQQHMLQLTVWGIKQLQARVLAVESYLKDQQLLGIWGCSGKHICTTTVP

SEQ ID NO: 124 - protein, 53 aa, Human immunodeficiency virus
EYKRLLRQRKIDWLIDRIRERAEDSGNESEGDTEELSTLVEMEPDNFRNDNDM

SEQ ID NO: 125 - protein, 56 aa, Human immunodeficiency virus
WETLKYLGSLVQYWGLELKKSAINLLNTTAIVVGEGTDRFIELIQRIWRAFCNIPR

SEQ ID NO: 126 - protein, 56 aa, Human immunodeficiency virus; identical to SEQ ID NO: 49

SEQ ID NO: 127 - protein, 56 aa, Human immunodeficiency virus
NTSWSNKSFNEIWDNMTWMEWEREINNYTNLIYNLIEESQNQQEKNEQDLLALDKW

SEQ ID NO: 128 - protein, 56 aa, Human immunodeficiency virus
SGIVQQQNNLLRAIEAQQHLLQLTVWGIKQLQARVLAVERYLRDQQLLGIWGCSGK

SEQ ID NO: 129 - protein, 56 aa, Human immunodeficiency virus
IYPGIKVRQLCKLLRGTKALTEVIPLTEEAELELAENREILKEPVHGVYYDPSKDL

SEQ ID NO: 130 - protein, 56 aa, Human immunodeficiency virus
NLTDNTKNIIVQLNETVTINCTRPGNNTRRGIHFGPGQALYTTGIVGDIRRAYCTI

SEQ ID NO: 131 - protein, 56 aa, Human immunodeficiency virus
GPGTASQRRNRRRRQRRRWLRLVALADKLYTFPDPPTDSPLDRAIQDLQRLTIHEL

SEQ ID NO: 132 - protein, 56 aa, Human immunodeficiency virus
YRLKHLVWASRELERFALNPGLLETPEGCLQIIEQIQPAIKTGTEELKSLFNLVAV

SEQ ID NO: 133 - protein, 56 aa, Human immunodeficiency virus
GIVQQQSNLLRAIEAQQHLLQLTVWGIKQLQSRVLAIERYLKDQQLLGIWGCSGKL

SEQ ID NO: 134 - protein, 56 aa, Human immunodeficiency virus
EGHLARNCRAPRKKGCWKCGKEGHQMKDCSERQANFLGKFWPLNKERPGNFLQNRP

SEQ ID NO: 135 - protein, 56 aa, Human immunodeficiency virus
FNCGKEGHIARNCKAPRKKGCWKCGREGHQMKDCTERQANFLREDLAFPQGKAREF

SEQ ID NO: 136 - protein, 56 aa, Human immunodeficiency virus
LICTTTVPWNTSWSNKSLNEIWDNMTWMKWEREIDNYTHIIYSLIEQSQNQQEKNE

SEQ ID NO: 137 - protein, 56 aa, Human immunodeficiency virus
NFTNNAKIIIVQLNKSVEINCTRPNNNTRNRISIGPGRAFHTTKQIIGDIRQAHCN

SEQ ID NO: 138 - protein, 56 aa, Human immunodeficiency virus
WNSSWSNKSLEEIWNNMTWIEWEREIDNYTGVIYSLIENSQIQQEKNEQDLLQLDK

SEQ ID NO: 139 - protein, 56 aa, Human immunodeficiency virus
TAVPWNASWSNKSLEEIWDNMTWMEWEREIDNYTSLIYTLIEESQNQQEKNEQELL

SEQ ID NO: 140 - protein, 53 aa, Human immunodeficiency virus
EYKKLVRQRKINRLYKRISERAEDSGNESEGDAEELAALGEVGPFIPGDINNL

SEQ ID NO: 141 - protein, 56 aa, Human immunodeficiency virus
HMGAASITLTVQARQLLSGIVQQQNNLLRAIEAQQHLLQLTVWGIKQLQARVLAVE

SEQ ID NO: 142 - protein, 56 aa, Human immunodeficiency virus
VPWNASWSNKSLNMIWNNMTWMQWEREIDNYTGIIYNLLEESQNQQEKNEQELLEL

SEQ ID NO: 143 - protein, 56 aa, Human immunodeficiency virus
YKLKHVVWASRELERFAINPGLLETSEGCRQILGQLQPSLQTGSEERKSLYNTVAT

SEQ ID NO: 144 - protein, 56 aa, Human immunodeficiency virus
KWEREIDNYTHIIYSLIEQSQNQQEKNEQELLALDKWASLWNWFDITKWLWYIKIF

SEQ ID NO: 145 - protein, 56 aa, Human immunodeficiency virus
ATLEEMMTACQGVGGPSHKARVLAEAMSQATNTAIMMQKSNFKGQRRIVKCFNCGK

SEQ ID NO: 146 - protein, 56 aa, Human immunodeficiency virus
AKAYEREAHNVWATHACVPTDPNPQEVFLKNVTENFDMWKNNMVEQMHTDIISLWD

SEQ ID NO: 147 - protein, 56 aa, Human immunodeficiency virus; identical to SEQ ID NO: 118

SEQ ID NO: 148 - protein, 56 aa, Human immunodeficiency virus
GGDMRDNWRSELYKYKVVKIEPLGVAPTKAKRRVVQREKRAVGIGALFLGFLGAAG

SEQ ID NO: 149 - protein, 56 aa, Human immunodeficiency virus
GIQRGWEILKYLGGLVQYWSLELKKSAISLFDTIAIAVAEGTDRIIEVIQGIWRAI

SEQ ID NO: 150 - protein, 52 aa, Human immunodeficiency virus
IRKIRRQRKIDRLIDRIRERAEDSGNESEGDTEELSKLVEMGHDAPWDVDDL

SEQ ID NO: 151 - protein, 52 aa, Human immunodeficiency virus
YKKLVRQKKIDRLIERIGERAEDSGNESDGDTEELSKLMEMGHLNLGYVADL

SEQ ID NO: 152 - protein, 56 aa, Human immunodeficiency virus
NAKNIIAHLNESVKITCARPYQNTRQRTPIGLGQSLYTTRSRSIIGQAHCNISRAQ

SEQ ID NO: 153 - protein, 56 aa, Human immunodeficiency virus
VQARNLLSGIVQQQSNLLKAIEAQQHLLQLTVWGIKQLQARILAVERYLKDQQLLG

SEQ ID NO: 154 - protein, 56 aa, Human immunodeficiency virus
IDIIATDIQTRELQKQITKIQNFRVYYRDSRDPIWKGPAKLPWKGEGAVVIQDNSE

SEQ ID NO: 155 - protein, 30 aa, Human immunodeficiency virus
DSGNESGGDTEELETMVDMGHLRLLDGNDL

SEQ ID NO: 156 - protein, 56 aa, Human immunodeficiency virus
PGAMQNCSFNMTTEVRDKKLKLSALFYRLDIVPIGNNNSSEYRLINCNTSTITQAC

SEQ ID NO: 157 - protein, 56 aa, Human immunodeficiency virus
NNTTLAPNVTISEEMKNCSFNITTEIRDKQKKEYALFYKLDVVQINNSNTSYRLIN

SEQ ID NO: 158 - protein, 56 aa, Human immunodeficiency virus
WGCAFRQVCHTTVPWVNDTLTPDWNNMTWQEWEQRIRNLEANISESLEQAQIQQEK

SEQ ID NO: 159 - protein, 56 aa, Human immunodeficiency virus
CSERQANFLGKFWPLNKERPGNFLQNRPEPTAPPAESFGFGEKITPSLRQEMKDQE

SEQ ID NO: 160 - protein, 56 aa, Human immunodeficiency virus
NNSINSANDEMKNCSFNITTELRDKKRKAYALFYKLDIVPLNNGSTDYRLINCNTS

SEQ ID NO: 161 - protein, 53 aa, Human immunodeficiency virus
EYRRIKRQRKIDCLIDRIRERAEDSGNESEGEREELSKLVEMGHHAPWDVDDL

SEQ ID NO: 162 - protein, 56 aa, Human immunodeficiency virus
NFTDNAKTIIVHLNESVQINCTRPNYNKRKRIHIGPGRAFYTTKNIIGTIRQAHCN

SEQ ID NO: 163 - protein, 56 aa, Human immunodeficiency virus
ILNGSLNTDGIVIRNDSHSNLLVQWNETVPINCTRPGNNTGGQVQIGPAMTFYNIE

SEQ ID NO: 164 - protein, 56 aa, Human immunodeficiency virus
LGIWGCSGKLICTTTVPWNTSWSNKSLDKIWNNMTWMEWEREIDNYTSLIYTLLEE

SEQ ID NO: 165 - protein, 56 aa, Human immunodeficiency virus
GAASLTLTVQARQLLSGIVQQQNNLLRAIEAQQHLLQLTVWGIKQLQARVLAVERY

SEQ ID NO: 166 - protein, 56 aa, Human immunodeficiency virus
KSQSDIWDKMTWLEWDKEVSNYTQVIYNLIEESQTQQEINERDLLALDKWANLWNW

SEQ ID NO: 167 - protein, 56 aa, Human immunodeficiency virus
PGASLEEMMTACQGVGGPAHKARVLAEAMSQVNNTTIMMQKSNFKGPKRAIKCFNC

SEQ ID NO: 168 - protein, 56 aa, Human immunodeficiency virus
SLNEIWDNMTWMQWEREIDNYTHLIYTLIEESQNQQEKNEQELLELDKWAGLWSWF

SEQ ID NO: 169 - protein, 56 aa, Human immunodeficiency virus
ERIIDIIASDIQTKELQKQIIKIQNFRVYYRDSRDPIWKGPAKLLWKGEGAVVIQD

SEQ ID NO: 170 - protein, 56 aa, Human immunodeficiency virus
VKTIIVQLNESVEINCTRPNNNTRKRITMGPGRVYYTTGQIIGDIRRAHCNLSRSK

SEQ ID NO: 171 - protein, 56 aa, Human immunodeficiency virus
KIEPLGVAPTKAKRRVVQREKRAVGLGALFLGFLGAAGSTMGAASITLTVQARQLL

SEQ ID NO: 172 - protein, 56 aa, Human immunodeficiency virus
NPTSSSWGMMEKGEIKNCSFYITTSIRNKVKKEYALFNRLDVVPIENTNNTKYRLI

SEQ ID NO: 173 - protein, 56 aa, Human immunodeficiency virus
NVDTEMKEEIKNCSYNMTTELRDKQRKIYSLFYRLDIVPIGGNSSNGDSSKYRLIN

SEQ ID NO: 174 - protein, 56 aa, Human immunodeficiency virus
AKAYETEVHNVWATHACVPTDPNPQEVVLENVTENFNMWKNNMVEQMHTDIISLWD

SEQ ID NO: 175 - protein, 56 aa, Human immunodeficiency virus
ASWSNKSLEQIWNNMTWMEWDREINNYTSLIHSLIEESQNQQEKNEQELLELDKWA

SEQ ID NO: 176 - protein, 56 aa, Human immunodeficiency virus
MSERADEEGLQGKLRLLRLLHQTNPYPQGPGTASQRRNRRRRRRRQWLRLVALANK

SEQ ID NO: 177 - protein, 56 aa, Human immunodeficiency virus
YRLKHLVWASRELERFALNPDLLETADGCQQILGQLQPALKTGTEDLQSLYNTIAV

SEQ ID NO: 178 - protein, 56 aa, Human immunodeficiency virus
YRLKHLVWASRELEKFTLNPGLLETAEGCQQILGQLQPALQTGTEELRSLYNTVAV

SEQ ID NO: 179 - protein, 56 aa, Human immunodeficiency virus
GKLICTTNVPWNSSWSNKSQSEIWDNMTWMEWDKQISNYTEEIYRLLEVSQTQQEK

SEQ ID NO: 180 - protein, 56 aa, Human immunodeficiency virus
KIKELREHLLKWGFTTPDKKHQKEPPFLWMGYELHPDKWTVQPIQLPEKEDWTVND

SEQ ID NO: 181 - protein, 53 aa, Human immunodeficiency virus
YRKILRQRKIDRLIDRLIERAEDSGNESEGEISALVEMGVEMGHHAPWDVDDL

SEQ ID NO: 182 - protein, 56 aa, Human immunodeficiency virus
NAKIIIVQLVKPVNITCMRPNNNTRKSISIGPGRAFFATGDIIGDIRQAHCNVSRT

SEQ ID NO: 183 - protein, 56 aa, Human immunodeficiency virus
IERYLRDQQILSLWGCSGKTICYTTVPWNETWSNNTSYDTIWNNLTWQQWDEKVRN

SEQ ID NO: 184 - protein, 56 aa, Human immunodeficiency virus
PTRAKRRVVEREKRAIGLGAVFLGFLGAAGSTMGAVSVALTGQARQLLSGIVQQQN

SEQ ID NO: 185 - protein, 56 aa, Human immunodeficiency virus
TGDTPINIFGRNILTALGMSLNLPVAKIEPIKVTLKPGKDGPRLKQWPLTKEKIEA

SEQ ID NO: 186 - protein, 56 aa, Human immunodeficiency virus
TQLLLNGSLAEEEIVIRSENFTNNAKTIIVQLNESVVINCTRPNNNTRKSINIGPG

SEQ ID NO: 187 - protein, 56 aa, Human immunodeficiency virus
ASLEEMMTACQGVGGPSHKARILAEAMSQVTNPVVMMQKGNFKGHRKIVKCFNCGK

SEQ ID NO: 188 - protein, 56 aa, Human immunodeficiency virus; identical to SEQ ID NO: 167

SEQ ID NO: 189 - protein, 56 aa, Human immunodeficiency virus
KDQQLLGFWGCSGKLICTTTVPWNASWSNKSLDDIWNNMTWMQWEREIDNYTSLIY

SEQ ID NO: 190 - protein, 56 aa, Human immunodeficiency virus
IPLGEARLVVTTYWGLHTGEKEWHLGQGVSIEWRKRRYSTQVDPGLADQLIHMYYF

SEQ ID NO: 191 - protein, 56 aa, Human immunodeficiency virus
YRLKHLVWASRELERFALNPNLLETVEGCRQIIRQLQPSLQTGSEELRSLFNTVAT

SEQ ID NO: 192 - protein, 56 aa, Human immunodeficiency virus
TTAVPWNASWSNKTLDQIWNNMTWMEWDREIDNYTHLIYTLIEESQNQQEKNQQEL

SEQ ID NO: 193 - protein, 56 aa, Human immunodeficiency virus
IWENMTWMQWEKEISNHTSTIYRLIEESQIQQEKNEQDLLALDKWASLWNWFDISN

SEQ ID NO: 194 - protein, 56 aa, Human immunodeficiency virus
AKAYDTEVHNVWATHACVPTDPNPQEVVLGNVTENFNMWKNNMVDQMHEDIISLWD

SEQ ID NO: 195 - protein, 56 aa, Human immunodeficiency virus
AKAYEKEVHNVWATHACVPTDPNPQEVEMENVTENFNMWKNNMVEQMHTDIISLWD

SEQ ID NO: 196 - protein, 56 aa, Human immunodeficiency virus
VVKIEPLGIAPTKTRRRVVEREKRAVGMGASFLGFLGAAGSTMGAASITLTVQARQ

SEQ ID NO: 197 - protein, 56 aa, Human immunodeficiency virus
STMGARSMTLTVQARQLLSGIVQQQNNLLRAIEAQQHLLQLTVWGIKQLQARILAV

SEQ ID NO: 198 - protein, 56 aa, Human immunodeficiency virus
EIVTHSFNCGGEFFYCNSTQLFTWNDTRKLNNTGRNITLPCRIKQIINMWQEVGKA

SEQ ID NO: 199 - protein, 56 aa, Human immunodeficiency virus
SSNWKEMDRGEIKNCSFKVTTSIRNKMQKEYALFYKLDVVPIDNDNTSYKLINCNT

SEQ ID NO: 200 - protein, 56 aa, Human immunodeficiency virus
MGAASITLTVQARQLLSGIVQQQSNLLRAIQAQQHMLQLTVWGIKQLQARVLAVER

SEQ ID NO: 201 - protein, 56 aa, Human immunodeficiency virus
LICTTNVRWNSSWSNKSYDDIWDNMTWMQWEKEIDNYTKTIYSLIEDAQNQQERNE

SEQ ID NO: 202 - protein, 56 aa, Human immunodeficiency virus
QQLLGIWGCSGKLICTTTVPWNSSWSNRSLNDIWQNMTWMEWEREIDNYTGLIYRL

SEQ ID NO: 203 - protein, 56 aa, Human immunodeficiency virus
QNNLLRAIEAQQHLLQLTVWGIKQLQARVLAVERYLRDQQLLGIWGCSGKLICTTT

SEQ ID NO: 204 - protein, 53 aa, Human immunodeficiency virus
EYRKILRQRKIDRLINRITERAEDSGNESDGDQEELSALVERGHLAPWDVDDL

SEQ ID NO: 205 - protein, 56 aa, Human immunodeficiency virus
YTSVKWNTSWSGRYNDDSIWDNLTWQQWDQHINNVSSIIYDEIQAAQDQQEKNVKA

SEQ ID NO: 206 - protein, 56 aa, Human immunodeficiency virus
NMTWMEWEKEIENYTNTIYTLIEESQIQQEKNEQELLELDKWASLWNWFGITKWLW

SEQ ID NO: 207 - protein, 54 aa, Human immunodeficiency virus
EYRKLLRQRKIDRLIDRIRERAEDSGNESEGDQEELSALVEMGGHDAPWDIDDL

SEQ ID NO: 208 - protein, 56 aa, Human immunodeficiency virus
DNWRSELYKYKVVKIEPLGVAPTKAKRRVVAREKRAIGMGAFFLGFLGAAGSTMGA

SEQ ID NO: 209 - protein, 56 aa, Human immunodeficiency virus
AKAYDTEVHNVWATHACVPTDPNPQEVVLENVTEDFNMWKNNMVEQMQEDVINLWD

SEQ ID NO: 210 - protein, 56 aa, Human immunodeficiency virus
MGNKWSKGWPAVRERIRQTPPAPPAAEGVGAASQDLAKHGAISSSNTATNNPDCAW

SEQ ID NO: 211 - protein, 56 aa, Human immunodeficiency virus
IVQLNESVVINCTRPNNNTRKSINIGPGRALYTTGEIIGDIRQAHCNLSKTQWENT

SEQ ID NO: 212 - protein, 56 aa, Human immunodeficiency virus
MTWMEWEREIDNYTNLIYTLIEESQNQQEKNEQELLELDKWASLWNWFDISKWLWY

SEQ ID NO: 213 - protein, 56 aa, Human immunodeficiency virus
TKILEPFRTKNPEIVIYQYMDDLYVGSDLEIGQHRTKIEELREHLLKWGFTTPDKK

SEQ ID NO: 214 - protein, 56 aa, Human immunodeficiency virus
MENRWQVLIVWQVDRQKVKAWNSLVKYHKYRSRKTENWWYRHHYESRNPRVSSSVY

SEQ ID NO: 215 - protein, 56 aa, Human immunodeficiency virus
KSMDQIWNNMTWMEWEREIDNYTSLIYNLIEESQNQQEKNEQELLELDKWASLWNW

SEQ ID NO: 216 - protein, 56 aa, Human immunodeficiency virus
ELYKYKVVKIEPLGVAPTKAKRRVVQREKRAVGVIGAMFLGFLGAAGSTMGAASIT

SEQ ID NO: 217 - protein, 56 aa, Human immunodeficiency virus
WRSELYKYKVIKIEPLGIAPTKAKRRVVQREKRAVGIVGAMFLGFLGAAGSTMGAV

SEQ ID NO: 218 - protein, 56 aa, Human immunodeficiency virus; identical to SEQ ID NO: 100

SEQ ID NO: 219 - protein, 56 aa, Human immunodeficiency virus
DQAQLNSWGCAFRQVCHTTVPWANESLTPDWNNMTWQEWEQKVRYLEANISQSLEE

SEQ ID NO: 220 - protein, 56 aa, Human immunodeficiency virus
YRMKHLVWASRELERFACNPGLMDTADGCAKLLNQLEPALKTGSEELRSLYNALAV

SEQ ID NO: 221 - protein, 56 aa, Human immunodeficiency virus
AKSYEKEAHNVWATHACVPTDPNPQEVVLENVTERFNMWENNMVEQMHTDIISLWD

SEQ ID NO: 222 - protein, 56 aa, Human immunodeficiency virus
AKAYDTEVHNVWATHACVPTDPNPQEIVLENVTENFNMWKNNMVEQMHEDIISLWD

SEQ ID NO: 223 - protein, 56 aa, Human immunodeficiency virus
DMRDNWRSELYKYKVVKIEPLGVAPTEARRRVVEREKRAVGMGAFFLGFLGAAGST

SEQ ID NO: 224 - protein, 56 aa, Human immunodeficiency virus
IPLGEARLVIKTYWGLHTGEREWHLGQGVSIEWRKRRYSTQVDPGLADQLIHMYYF

SEQ ID NO: 225 - protein, 56 aa, Human immunodeficiency virus
KALGRGASIEEMMTACQGVGGPSHKARVLAEAMSQVTNASAAIMMQKGNFKGPRRT

SEQ ID NO: 226 - protein, 56 aa, Human immunodeficiency virus
NETWSNNTSYDTIWNNLTWQQWDEKVRNYSGVIFGLIEQAQEQQNTNEKSLLELDQ

SEQ ID NO: 227 - protein, 56 aa, Human immunodeficiency virus
GGGDMRDNWRSELYKYKVVKIEPLGVAPTRAKRRVVQREKRAVGIGAVFLGFLGAA

SEQ ID NO: 228 - protein, 56 aa, Human immunodeficiency virus
TVQARQLLSGIVQQQNNLLRAIEAQQQMLQLTVWGIKQLRARVLAVERYLRDQQLL

SEQ ID NO: 229 - protein, 56 aa, Human immunodeficiency virus
AKAYEREVHNVWATYACVPTDPSPQELVLGNVSEKFNMWKNNMVDQMHEDIISLWD

SEQ ID NO: 230 - protein, 56 aa, Human immunodeficiency virus
MSILQIVAIVAIIVALILAIVVWTIVYIEYKRLLRQRKIDWLIDRIRERAEDSGNE

SEQ ID NO: 231 - protein, 53 aa, Human immunodeficiency virus
EYRKILRQRKIDRLIDKIRERAEDSGNESEGDQEELSALVERGHLAPWDINDL

SEQ ID NO: 232 - protein, 56 aa, Human immunodeficiency virus
REIKEALKHFDPRLLITLGNYIYARHGDTLEGARGLIRILQRALLLHFRAGCGRSR

SEQ ID NO: 233 - protein, 56 aa, Human immunodeficiency virus
MGGKWSKSSVVGWPAVRERMRRAEPAADGVGAASRDLEKHGAITSSNTAANNAACA

SEQ ID NO: 234 - protein, 56 aa, Human immunodeficiency virus
VRVIHTDNGSNFTSNAVKAACWWAGIQQEFGIPYNPQSQGVVESMNKELKKIIGQV

SEQ ID NO: 235 - protein, 56 aa, Human immunodeficiency virus
MGKIWSKSSLVGWPEIRERIRRQTPEPAVGVGAVSQDLANRGAITTSNTKDNNQTV

SEQ ID NO: 236 - protein, 56 aa, Human immunodeficiency virus
MGGTMSKCSPVGWPAIRERIRRAAPAAEGVGAASRDLDKYGALTSSNTPANNPDCA

SEQ ID NO: 237 - protein, 56 aa, Human immunodeficiency virus
AKAYKKEAHNIWATHACVPTDPNPQEIELENVTENFNMWKNNMVEQMHEDIISLWD

SEQ ID NO: 238 - protein, 56 aa, Human immunodeficiency virus
YALKHLIWASRELERFTLNPGLLETSEGCKQIIGQLQPSIQTGSEEIRSLYNTVAT

SEQ ID NO: 239 - protein, 56 aa, Human immunodeficiency virus
GERLIDILASQIQTTELQKQILKINNFRVYYRDSRDPIWKGPAQLLWKGEGAVVIQ

SEQ ID NO: 240 - protein, 56 aa, Human immunodeficiency virus
KAKRRVVQREKRAVGIGALFLGFLGAAGSTMGARSMTLTVQARQLLSGIVQQQNNL

SEQ ID NO: 241 - protein, 56 aa, Human immunodeficiency virus
YRLKHLVWASRELERYACNPGLLETAEGTEQLLQQLEPALKTGSEDLKSLWNAIAV

SEQ ID NO: 242 - protein, 56 aa, Human immunodeficiency virus
IETVPVKLKPGMDGPKIKQWPLTEEKIKALTQICAEMEEEGKISRVGPENPYNTPV

SEQ ID NO: 243 - protein, 56 aa, Human immunodeficiency virus
QAIEDVWRLFETSIKPCVKLTPLCVAMNCNITSGTTATPSPPNITIIDENSTCIGD

SEQ ID NO: 244 - protein, 56 aa, Human immunodeficiency virus
NWRSELYKYKVVKIEPLGVAPTKAKRRVVQREKRAVGAIGAMFLGFLGAAGSTMGA

SEQ ID NO: 245 - protein, 56 aa, Human immunodeficiency virus
TETFRPGGGNMRDNWRSELYKYKVVKIELLGVAPTKAKRRVVQREKRAVGIGAVFL

SEQ ID NO: 246 - protein, 56 aa, Human immunodeficiency virus
SWVPAHKGIGGNEQVDKLVSSGIRKVLFLDGIDKAQEEHERYHSNWRAMASDFNLP

SEQ ID NO: 247 - protein, 56 aa, Human immunodeficiency virus
ARAYDTEVHNVWATHACVPTDPNPQEVVLGNVTENFNMWKNNMVEQMQEDIISLWD

SEQ ID NO: 248 - protein, 53 aa, Human immunodeficiency virus
EYRRIKKQRRIDCLLDRITERAEDSGNESEGDREKLSKLVEMGHHAPWDIDDL

SEQ ID NO: 249 - protein, 56 aa, Human immunodeficiency virus
RIEPVGVAPTKAKRRVVQRAKRAVGMGAVLFGFLGAAGSTMGAAAITLTAQARQLL

SEQ ID NO: 250 - protein, 56 aa, Human immunodeficiency virus
KWKPKMIGGIGGFIKVRQYDQIIIEICGKKAIGTVLVGPTPVNIIGRNMLTQLGRT

SEQ ID NO: 251 - protein, 53 aa, Human immunodeficiency virus
EIRKTLRQKKIDRLIDRIRERAEDSGNESDGDEEELSALVEMGHHAPWDVDDL

SEQ ID NO: 252 - protein, 56 aa, Human immunodeficiency virus
HQKEPPFLWMGYELHPDKWTVQPIQLPDKESWTVNDIQKLVGKLNWASQIYPGIKV

SEQ ID NO: 253 - protein, 52 aa, Human immunodeficiency virus
YRKILRQRKIDRLIDRIRERAEDSGNESEGDQEELSALVEMGHDAPWDIDDL

SEQ ID NO: 254 - protein, 56 aa, Human immunodeficiency virus
GIGGFINTKEYKDVEIEVVGKRVRATIMTGDTPINIFGRNILNTLGMTLNFPVAKI

SEQ ID NO: 255 - protein, 56 aa, Human immunodeficiency virus
EHRKRVELLREHLYQWGFTTPDKKHQKEPPFLWMGYELHPDKWTVQPIQLPDKEVW

SEQ ID NO: 256 - protein, 56 aa, Human immunodeficiency virus
KTIIGGGEVKNCSFNITTSIRDKVHKEYALFYKLDVVPIKSNNDSSTYTRYRLIHC

SEQ ID NO: 257 - protein, 56 aa, Human immunodeficiency virus
EKDLLALDKWENLWNWFNITNWLWYIKIFIMIVGGVIGLRIIFAVLSIVNRVRQGY

SEQ ID NO: 258 - protein, 56 aa, Human immunodeficiency virus
LFCASDAKAYDTEAHNVWATHACVPTDPNPQEVVLVNVTENFNMWKNNMVEQMHEN

SEQ ID NO: 259 - protein, 56 aa, Human immunodeficiency virus
SGKLICTTNVPWNSSWSNKSLEEIWGNMTWMEWEKEVSNYSKEIYRLIEDSQNQQE

SEQ ID NO: 260 - protein, 56 aa, Human immunodeficiency virus
TIVFIEYRKIRKEKKIEYLIDRIRERAEDSGNESEGDTGELAKLVEMGDFDPWVGD

SEQ ID NO: 261 - protein, 56 aa, Human immunodeficiency virus
THGIKPVVSTQLLLNGSLAEEEVIIRSSNFTNNAKIIIVQLNKSVEINCTRPNNNT

SEQ ID NO: 262 - protein, 53 aa, Human immunodeficiency virus
LIRKILRQRKIDRLIDRIRERAEDSGNESEGIRKELSALVEMGHDAPGDIDDL

SEQ ID NO: 263 - protein, 56 aa, Human immunodeficiency virus
QLLSGIVQQQSNLLRAIEAQQHLLQLTVWGIKQLQARVLAVERYLKDQQLLGLWGC

SEQ ID NO: 264 - protein, 56 aa, Human immunodeficiency virus
TRAKRRVVQREKRAVGIGAVFLGFLGAAGSTMGAAAVTLTVQARQLLPGIVQQQNN

SEQ ID NO: 265 - protein, 56 aa, Human immunodeficiency virus
RRGIHFGPGQALYTTGIVGDIRRAYCTINETEWDKTLQQVAVKLGSLLNKTKIIFN

SEQ ID NO: 266 - protein, 56 aa, Human immunodeficiency virus
AGSTMGAVSVALTGQARQLLSGIVQQQNNLLRAIEAQQHMLQLTVWGIKQLQARVL

SEQ ID NO: 267 - protein, 56 aa, Human immunodeficiency virus
WEQKVRYLEANISQSLEQAQIQQEKNMYELQKLNSWDVFTNWLDFTSWVRYIQYGV

SEQ ID NO: 268 - protein, 56 aa, Human immunodeficiency virus
EIIIRSENLTNNVKTIIVHLNESVEINCTRPDNKITRQSTPIGLGQALYTTRIKGD

SEQ ID NO: 269 - protein, 56 aa, Human immunodeficiency virus
AKSYKTEAHNIWATHACVPTDPNPQEIELENVTENFNMWRNNMVEQIHEDIISLWD

SEQ ID NO: 270 - protein, 56 aa, Human immunodeficiency virus
MGGKWSKSSVIGWPTVRERMRRAEPAADGVGAASQDLEKHGAITSSNTAATNADCA

SEQ ID NO: 271 - protein, 56 aa, Human immunodeficiency virus
MQWDREISNYTDIIYNLLEVSQNQQDKNEKDLLALDKWENLWNWFNITNWLWYIKI

SEQ ID NO: 272 - protein, 56 aa, Human immunodeficiency virus
LYKYKVVKIEPLGVAPTKAKRQVVQREKRAVGMGAMFLGFLGAAGSTMGAASITLT

SEQ ID NO: 273 - protein, 56 aa, Human immunodeficiency virus
GKEGHLARNCRAPRKKGCWKCGKEGHQMKDCTERQANFFRETLAFQQGKAREFPSE

SEQ ID NO: 274 - protein, 56 aa, Human immunodeficiency virus
HRAKIEELREHLLKWGLTTPDKKHQKEPPFLWMGYELHPDKWTVQPIQLPEKEDWT

SEQ ID NO: 275 - protein, 56 aa, Human immunodeficiency virus
LGPQATLEEMMTACQGVGGPSHKARVLAEAMSQATNSVTTAMMQRGNFKGPRKIIK

SEQ ID NO: 276 - protein, 56 aa, Human immunodeficiency virus
GLGTARQRRNRRRRWERRWKQILALADRIYTFPDPPADPPLDQTIQQLQGLTIQTL

SEQ ID NO: 277 - protein, 53 aa, Human immunodeficiency virus
EYRKILRQRKIDRLIDRIREREEDNGNESEGDQEELSALVEMGHHAPWNVDDL

SEQ ID NO: 278 - protein, 56 aa, Human immunodeficiency virus
EIFRPGGGDMRDNWRSELYKYKVVKIEPLGVAPTKAKRRVVQREKRAVGMLGAMFL

SEQ ID NO: 279 - protein, 56 aa, Human immunodeficiency virus
DKKFNGTGPCTNVSTVQCTHGIKPVVSTQLLLNGSLAEGEVVIRSENFTNNAKTII

SEQ ID NO: 280 - protein, 56 aa, Human immunodeficiency virus
AKAYKTEVHNVWAKHACVPTDPNPQEVLLENVTENFNMWKNNMVEQMHEDIISLWD

SEQ ID NO: 281 - protein, 56 aa, Human immunodeficiency virus
QVRQLLSGIVQQQSNLLRAIEAQQHMLQLTVWGIKQLQTRVLAIERYLRDQQLLGI

SEQ ID NO: 282 - protein, 56 aa, Human immunodeficiency virus
AKAYDTEVHNVWATQACVPTDPNPQEVELVNVTENFNMWKNNMVEQMHEDIISLWD

SEQ ID NO: 283 - protein, 56 aa, Human immunodeficiency virus
TWMEWEREIDNYTGLIYSLIEESQIQQEKNEKELLELDKWASLWNWFSITKWLWYI

SEQ ID NO: 284 - protein, 56 aa, Human immunodeficiency virus; identical to SEQ ID NO: 241

SEQ ID NO: 285 - protein, 56 aa, Human immunodeficiency virus
TICYMRTCNTSVIQESCDKHYWDSLRFRYCAPPGYALLRCNDTNYSGFMPKCSKVV

SEQ ID NO: 286 - protein, 56 aa, Human immunodeficiency virus
TERQANFFRENLAFQQGEARKFPSEQTRANSPTSRELRVQRGDNPLSEAGAERRGT

SEQ ID NO: 287 - protein, 56 aa, Human immunodeficiency virus
MRDNWISELYKYKVVRIEPLGVAPTKAKRRVVEREKRAIGLGAMFLGFLGAAGSTM

SEQ ID NO: 288 - protein, 56 aa, Human immunodeficiency virus
IKSLGVAPTRARRRVVEREKRAVGLGAVFLGFLGAAGSTMGAASITLTAQVRQLLS

SEQ ID NO: 289 - protein, 56 aa, Human immunodeficiency virus
RDGGNDNNTRTEETFRPGGGDMRDNWRSELYKYKVVQIEPLGIAPTRARRRVVQRE

SEQ ID NO: 290 - protein, 56 aa, Human immunodeficiency virus
TNGTEIFRPGGGDMRDNWRSELYKYKVVKIEPLGVAPTKAKRRVVQREKRAVGLGA

SEQ ID NO: 291 - protein, 56 aa, Human immunodeficiency virus
SLNDIWQNMTWMEWEREIDNYTGLIYRLIEESQTQQEKNEQELLELDKWASLWNWF

SEQ ID NO: 292 - protein, 56 aa, Human immunodeficiency virus
TRDGGNNNETTDTEIFRPGGGNMRDNWRSELYKYKVVKIEPLGVAPTKAKRRVVQR

SEQ ID NO: 293 - protein, 56 aa, Human immunodeficiency virus
MRDNWRSELYKYKVVTIEPLGVAPTKAKRRVVQREKRAAIGALFLGFLGAAGSTMG

SEQ ID NO: 294 - protein, 56 aa, Human immunodeficiency virus
GYSAGERIIDMIATDIQTKELQKQITKIQNFRVYYRDNRDPIWKGPAKLLWKGEGA

SEQ ID NO: 295 - protein, 53 aa, Human immunodeficiency virus
EYRKILRQRKIDRLFDRIREKAEDSGNESERDQEELSALVEMGHLAPWDVDDL

SEQ ID NO: 296 - protein, 56 aa, Human immunodeficiency virus
GPGATLEEMMTACQGVGGPSHKARVLAEAMSQASGAAAAAIMMQKSNFKGPRRIIK

SEQ ID NO: 297 - protein, 56 aa, Human immunodeficiency virus
NWRSELYKYKVVKIEPLGVAPTKAKRRVVEREKRAIGLGAMFLGFLGAAGSTMGAA

SEQ ID NO: 298 - protein, 56 aa, Human immunodeficiency virus
QLMSGIVHQQNNLLRAIEAQQHLLQLTVWGIKQLQARVLAVERYLRDQQLLGIWGC

SEQ ID NO: 299 - protein, 56 aa, Human immunodeficiency virus
RSELYKYKVVQIEPLGVAPTRAKRRVVEREKRAIGLGAMFLGFLGAAGSTMGARSV

SEQ ID NO: 300 - protein, 56 aa, Human immunodeficiency virus
LQSRPEPTAPPFLQSRPEPTAPPEESFRSGVETTTPPQKQEPIDKELYPLTSLRSL

SEQ ID NO: 301 - protein, 56 aa, Human immunodeficiency virus
CRDKTFTGTGPCHNVSTVQCTHGIKPVVSTQLLLNGSIAEGETIIRFENLTNNAKI

SEQ ID NO: 302 - protein, 56 aa, Human immunodeficiency virus
LKKIIGQVRDQAEHLKTAVQMAVFIHNFKRKGGIGGYSAGERIVDIIATDIQTKEL

SEQ ID NO: 303 - protein, 56 aa, Human immunodeficiency virus
TWMEWEKEVSNYSKEIYRLIEDSQNQQEKNEQELLALDKWASLWNWFDITQWLWYI

SEQ ID NO: 304 - protein, 56 aa, Human immunodeficiency virus
CLFSYHRLRDLILIAARAVELLGRSSLRGIQRGWEILKYLGGLVQYWSLELKKSAI

SEQ ID NO: 305 - protein, 56 aa, Human immunodeficiency virus
AKAYDTEVHNVWATHACVPTDPNPQEVKLENVTENFNMWKNNMVEQMHEDIISLWD

SEQ ID NO: 306 - protein, 56 aa, Human immunodeficiency virus
KKAQQAAADTGNNSQVSQNYPIVQNLQGQMVHQAISPRTLNAWVKVIEEKAFSPEV

SEQ ID NO: 307 - protein, 56 aa, Human immunodeficiency virus
ATEKLWVTVYYGVPVWKEATTTLFCASDAKAYDTEVHNVWATQACVPTDPNPQEVE

SEQ ID NO: 308 - protein, 56 aa, Human immunodeficiency virus
SELYKYKVVKIEPLGVAPTKAKRRVVQREKRAVGLGALFLGFLGAAGSTMGAASLT

SEQ ID NO: 309 - protein, 56 aa, Human immunodeficiency virus
YKVVKIEPLGVAPTKAKRRVVQREKRAVTLGAMFLGFLGAAGSTMGARSLTLTVQA

(19) United States
(12) Patent Application Publication
(10) Pub. No.: US 2022/0065857 A1
(43) Pub. Date: Mar. 3, 2022

(54) HIV SEROSIGNATURES FOR CROSS-SECTIONAL INCIDENCE ESTIMATION

(71) Applicant: THE JOHNS HOPKINS UNIVERSITY, Baltmore, MD (US)

(72) Inventors: Susan H. Eshleman, Lutherville, MD (US); Oliver Laeyendecker, Baltimore, MD (US); Harry B. Larman, Baltimore, MD (US); Kai Kammers, Baltimore, MD (US)

(21) Appl. No.: 17/312,916

(22) PCT Filed: Dec. 9, 2019
(86) PCT No.: PCT/US2019/065132

§ 371 Date: Jun. 10, 2021

Related U.S. Application Data
(60) Provisional application No. 62/778,347, filed on Dec. 12, 2018.

(51) Int. Cl. G01N 33/569

(52) U.S. Cl. CPC G01N 33/56988; G01N 2333/16

(57) ABSTRACT

Described are methods for estimating the cross-sectional incidence or duration of infection of a virus. Method steps include obtaining a biological sample with antibodies from a subject having a viral infection. The biological sample is mixed with two or more epitopes or peptides from the proteins of a vims responsible for the viral infection. The amount of antibody binding to the epitopes or peptides is quantified and the cross-sectional incidence or duration of infection of a virus is estimated.

### REFERENCE TO RELATED APPLICATIONS

This application claims the benefit of U.S. Provisional Patent application 62/778,342, filed Dec. 12, 2018, which is hereby incorporated by reference for all purposes as if fully set forth herein.

### STATEMENT OF GOVERNMENTAL INTEREST

This invention was made with government support under grant nos. AI118633, AI068613, and AI095068 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Nov. 19, 2019, is named P15406-02_SL.txt and is 172,811 bytes in size.

### BACKGROUND OF THE INVENTION

Antibodies to HIV appear shortly after infection. The titer and avidity of anti-HIV antibodies generally increase over time, but may be impacted by antiretroviral treatment (ART), CD4 T cell decline, and other factors. The breadth and specificity of anti-HIV antibodies also evolve during the course of infection. A detailed understanding of the serologic response to HIV infection is helpful for understanding HIV immune containment and for vaccine development. Multiplexed immunoassays have been used to analyze the specificity of anti-HIV antibodies. These include a microarray assay composed of 15 recombinant HIV env protein targets and five gp41 peptide targets, and an assay based on the Luminex platform that includes six recombinant HIV protein targets. Phage display technology has also been used to screen HIV peptides for binding to immobilized antibodies (6).

HIV incidence is the rate at which new HIV infections occur in populations. While HIV prevalence measures overall disease burden, HIV incidence tracks the leading edge of the HIV/AIDS epidemic. Accurate HIV incidence estimates are critical for monitoring the epidemic, identifying populations at high risk of HIV acquisition, targeting prevention efforts, and evaluating interventions for HIV prevention. HIV incidence can be measured by evaluating HIV seroconversion in longitudinal cohorts and modeling trends in HIV prevalence; however, those approaches have significant practical and methodological limitations. An alternative approach is to use a cross-sectional survey to identify recent infections and estimate HIV incidence. Serologic (antibody-based) assays have been developed for cross-sectional HIV incidence estimation. These assays measure characteristics of the HIV antibody response such as the titer, class, and avidity of anti-HIV antibodies. The United States (US) Centers for Disease Control and Prevention (CDC) has developed two HIV incidence assays: the BED capture immunoassay (BED assay)³, which measures the proportion of antibody that is HIV-specific; and the newer Limiting Antigen Avidity assay (LAg assay), which measures antibody binding to a limited amount of a target antigen. Unfortunately, the serologic response to HIV infection is highly variable. Some HIV-infected individuals never attain a mature antibody response, and numerous factors, such as advanced HIV disease and viral suppression, can blunt the antibody response. Performance of serologic incidence assays also varies by geographic region and in different sub-populations, reflecting differences in HIV subtype and other factors. The highest levels of misclassification are seen with subtype D HIV, which is associated with reduced serologic responses to HIV infection. While serologic HIV incidence assays at first seemed promising, it is now clear that these assays provide inaccurate incidence estimates in some settings and populations because of sample misclassification.

### SUMMARY OF THE INVENTION

One embodiment of the present invention is a method of estimating the cross-sectional incidence or duration of infection of a virus. Method steps include obtaining a biological sample that contains antibodies from a subject who has one or more viral infections; mixing the biological sample with two or more epitopes or peptides from the proteins of viruses responsible for the viral infection; quantifying the amount of antibody binding to the epitopes or peptides; and estimating the cross-sectional incidence or duration of infection for one or more of the viruses. The methods of the present invention estimate the cross-sectional incidence or duration of infection for a virus that infects mammals, including HIV and EBV, as examples. In addition, the epitopes or peptides of the present invention may be derived from, or expressed in, a phage immunoprecipitation sequencing system (PhIP-Seq or VirScan). The epitopes or peptides of the present invention may be modified by site-directed mutagenesis using alanine substitution, or another method, to alter the amino acid sequence of the peptides. The epitopes or peptides of the present invention may be synthesized chemically or used in a biologic system. For example, the epitopes or peptides of the present invention, including SEQ ID:1 to SEQ ID:309, may be used in a assay system including enzyme immunoassay, chemiluminescent assay, microparticle bead assay, electrochemiluminescent assay, and a combination thereof. The assay systems detect and/or quantify binding of antibodies to one or more epitopes or peptides, either individually or in a multiplex (multi-assay) format.

Another embodiment of the present invention is a method of estimating or calculating the cross-sectional incidence or duration of infection of HIV, including HIV subtype C and HIV subtype D infections. HIV proteins including gp41, gp120, gag, and pol, as examples, are used in methods of the present invention. In addition, the plurality of epitopes or peptides may be selected from the group consisting of SEQ ID:1 to SEQ ID:309. Alternatively, the plurality of epitopes or peptides may be selected from the group consisting of SEQ ID:1 to SEQ ID:309 in the range of 2 to 200, 3 to 150, 4 to 125, 5 to 100, 7 to 100, 10 to 100, 4 to 20, 4 to 30, 4 to 50, 8 to 60 or 10 to 70 epitopes or peptides. Alternatively, the epitopes or peptides of the present invention may comprise SEQ ID:3, SEQ ID:22, SEQ ID:159 and SEQ ID:180.

### Definition of Terms

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The term “antibody,” as used in this disclosure, refers to an immunoglobulin or a fragment or a derivative thereof, and encompasses any polypeptide comprising an antigen-binding site, regardless of whether it is produced in vitro or in vivo. The term includes, but is not limited to, polyclonal, monoclonal, monospecific, polyspecific, non-specific, humanized, single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind, for example, PD-L1, specifically. Typically, such fragments would comprise an antigen-binding domain.

The terms “antigen-binding domain,” “antigen-binding fragment,” and “binding fragment” refer to a part of an antibody molecule that comprises amino acids responsible for the specific binding between the antibody and the antigen. In instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}), however, it does not necessarily have to comprise both. For example, a so-called Fd antibody fragment consists only of a V_{H} domain, but still retains some antigen-binding function of the intact antibody.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

By “disease” is meant any condition or disorder that damages or interferes with the normal function of a cell, tissue, or organ. Examples of diseases include HIV.

The term “express” refers to the ability of a gene to express the gene product including for example its corresponding mRNA or protein sequence (s).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “gag” or “group-specific antigen” is a gene that codes for core structural proteins of a retrovirus. For example, HIV gag protein is encoded by the HIV gag gene, HXBE nucleotides 790-2292. One example of a HIV gag protein has a NCBI database accession number ASM60435.

By “gp 41” or “glycoprotein 41” is meant a subunit of the envelope protein complex of retroviruses, including human immunodeficiency virus (HIV). Gp41 is a transmembrane protein that contains several sites within its ectodomain that are required for infection of host cells. As a result of its importance in host cell infection, it has also received much attention as a potential target for HIV vaccines. One example of a HIV gp41 protein has a NCBI database accession number ASV70553.1.

By “gp120” or “Envelope glycoprotein GP120 is meant a glycoprotein exposed on the surface to a retrovirus envelope such as HIV. Gp120 is essential for virus entry into cells as it plays a vital role in attachment to specific cell surface receptors. One example of a HIV gp120 protein has a NCBI database accession number AAF69493.1.

By “immunoassay” is meant an assay that uses an antibody to specifically bind an antigen (e.g., a marker). The immunoassay is characterized by the use of specific binding properties of a particular antibody to isolate, target, and/or quantify the antigen.

By “incidence of infection” is meant the frequency of new infections occurring over a specified period of time (e.g., annual HIV incidence is the percentage of individuals who acquire HIV infection during one year).

The term, “obtaining” as in “obtaining an agent” includes synthesizing, purchasing, or otherwise acquiring the agent.

By “marker” is meant any protein or polynucleotide or antibody having an alteration in expression level or activity that is associated with a disease or disorder. The term “biomarker” is used interchangeably with the term “marker.”

The term “mAb” refers to monoclonal antibody. Antibodies may comprise without limitation whole native antibodies, bispecific antibodies; chimeric antibodies; Fab, Fab′, single chain V region fragments (scFv), fusion polypeptides, and unconventional antibodies.

The term “measuring” means methods which include detecting the presence or absence of marker(s) such as antibodies in the sample, quantifying the amount of marker(s) such as antibodies in the sample, and/or qualifying the type of biomarker or antibody. Measuring can be accomplished by methods known in the art and those further described herein, including but not limited to immunoassay. Any suitable methods can be used to detect and measure one or more of the markers described herein. These methods include, without limitation, ELISA and bead-based immunoassays (e.g., monoplexed or multiplexed bead-based immunoassays, magnetic bead-based immunoassays).

By “pol” is meant a DNA polymerase encoded by a gene in retroviruses, such as HIV. The pol protein is an enzyme that transcribes viral RNA into double-stranded DNA. One example of a HIV pol protein has a NCBI database accession number AAF35355.1.

The terms “polypeptide,” “peptide”, and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “reduces” is meant a negative alteration of at least 10%, 25%, 50%, 75%, or 100%.

A “reference” refers to a standard or control conditions such as a sample (human cells) or a subject that is a free, or substantially free, of an agent or disease.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

As used herein, the term “sensitivity” is the percentage of subjects with a particular disease.

As used herein, the term “specificity” is the percentage of subjects correctly identified as NOT having a particular disease i.e., normal or healthy subjects.

By “specifically binds” is meant an antibody that recognizes and binds a polypeptide of the invention such as a gp41 polypeptide, a gp120 polypeptide, a gag polypeptide, or a pol polypeptide, but which does not substantially recognize and bind other molecules in a sample, for example, a biological sample, which naturally includes a polypeptide of the invention.

As used herein, the term “subject” is intended to refer to any individual or patient to which the method described herein is performed. Generally, the subject is human, although as will be appreciated by those in the art, the subject may be an animal. Thus, other animals, including mammals such as rodents (including mice, rats, hamsters, guinea pigs, cats, dogs, rabbits, farm animals including cows, horses, goats, sheep, pigs, etc., and primates (including monkeys, chimpanzees, orangutans and gorillas) are included within the definition of subject.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### BRIEF DESCRIPTION OF THE DRAWINGS

FIG. 1A-1E. Antibody reactivity to peptides spanning the HIV proteome.

FIG. 1 illustrates the size and position of open reading frames (ORFs) in the HIV genome. Panels B-D are plotted relative to genomic coordinates for HIV (HXB2, NCBI #NC 001802), shown at the bottom of the figure. Panel B: The plot shows the number of peptide tiles encoded by the VirScan library at each position across the HIV genome. Panel C: The plot shows the average level of antibody binding (average z-score) for each peptide for the 403 samples in the discovery sample set; each dot represents antibody binding for a single peptide in the VirScan library. Panel D: The plot shows the percentage of study participants who had a high level of antibody binding for each peptide (z-score>10). Panel E: The figure shows a heat map of the level of antibody binding for peptides in the VirScan library as a function of duration of HIV infection. The position of peptides is shown on the x-axis; the duration of infection is shown on the y-axis. Z-scores are noted according to the color bar on the right; lighter colors (higher z-scores) indicate a higher level of antibody binding. For each sample, data are plotted in order of increasing z-scores, since many points were overlapping.

Abbreviations: ORF: open reading frame; mo: months; yr: years; kb: kilobases.

FIG. 2A-2C. Breadth of antibody reactivity

FIG. 2 illustrates information related to antibody breadth. Panel A: The relationships between peptides that were highly enriched (z-score>10) are displayed as a network graph; data are from a single representative sample. Peptides (nodes) are indicated by circles. Darker red color indicates peptides with higher z-scores. Overlapping peptides that share amino acid sequences form clusters in the graph; the position of the peptides in HIV proteins is noted for each cluster (the HIV protein is listed first, followed by numbers that represent the range of amino acid positions of the N-termini of peptides in the cluster). Peptides are linked (connected by lines) if they share an identical sequence of at least seven consecutive amino acids. In this case, network graph analysis of 573 reactive peptides identified 45 unique peptide specificities (circles outlined in black), corresponding to an antibody breadth value of 45. Panels B and C. Antibody breadth is plotted as a function of duration of HIV infection. The top two graphs (Panel B) show breadth data for HIV peptides; the bottom two graphs (Panel C) show breadth data for EBV peptides. Each line represents results from a single study participant. The two graphs on the left show data for participants who did not start antiretroviral treatment in the GS Study (no ART, N=33); the two graphs on the right show data for participants who reported starting antiretroviral treatment (ART, N=24). Data from samples collected after treatment initiation are shown in red (on ART). Dark blue lines indicate the locally-weighted regression (lowest) curves for all participants in each graph.

Abbreviations: Env: envelope; Pol: polymerase; Gag: group-specific antigen; Rev: HIV regulatory protein; Vpu: viral protein U; ART: antiretroviral treatment; mo: months; yr: years;

EBV: Epstein Barr virus.

FIG. 3. Relationship between changes in antibody breadth and time to ART.

FIG. 3 illustrates time-to-event (survival) analysis for the outcome of time from HIV infection to antiretroviral treatment initiation (time to ART), comparing participants with declining vs. stable or increasing antibody breadth (shown in red and blue, respectively). The change in antibody breadth was calculated for the time period between 9 months and 2 years after HIV infection, using samples collected closest to these dates. The median sample collection times were 0.8 years for the visit 9 months after infection (range 0.55-0.98 years) and 1.5 years for the visit 2 years after infection (range 1.26-3.12 years); the median time to ART initiation was 3.34 years (range 1.16-6.35 years). Data from two participants were removed for this analysis (one did not have viral load data and one started ART<2 years after HIV infection). The survival curves are based on estimated hazard ratios (lines) with 95% confidence intervals (shaded areas). The number of participants at risk (Number at risk; not yet on ART) at each time point is shown below the graph for each participant group.

Abbreviations: Ab: antibody; ART: antiretroviral therapy; Decr: decreasing antibody breadth; Non-Decr: stable or increasing antibody breadth.

FIG. 4A-4B. Association of antibody binding and the duration of HIV infection.

FIG. 4A illustrates data evaluating the association of antibody binding (normalized read counts) and the duration of HIV infection for 3,327 peptides in the VirScan library that had well-defined positions in the HIV genome. P-values were calculated using generalized estimation equations to account for the dependency between measurements over time from the same individual and were adjusted using the Bonferroni correction based on all 3,384 identified HIV peptides. The x-axis shows the position of each peptide and the y-axis shows the corresponding Bonferroni adjusted p-value. Black dots represent peptides where antibody binding was positively associated with the duration of infection (266 peptides with adjusted p-values<0.05); red dots represent peptides where antibody binding was negatively associated with the duration of infection (43 peptides with adjusted p-values<0.05). FIG. 4B shows the position of open reading frames (ORFs) in the HIV genome (reproduced from FIG. 1, Panel A).

FIG. 5A-5D: Use of a 4-peptide model to predict duration of HIV infection.

FIG. 5 illustrates data from the 4-peptide model. Four peptides were selected from the VirScan library that had the strongest independent association between antibody binding and the duration of HIV infection. This included two peptides that had increasing antibody binding over time, and two peptides that had decreasing antibody binding over time (Supplemental FIG. 2). Panels A-C: Data from these four peptides (normalized read counts) were summed to generate a composite antibody binding score for each of the 403 samples in the discovery sample set that was used to identify the four peptides (Table 1). The plots show the observed duration of HIV infection (y-axes) and the duration of HIV infection that was predicted using a simple linear regression model based on the composite antibody binding score for the four peptides (x-axes). In the graphs, each dot represents data from a single sample. The same data are plotted in Panels A-C. Red dots represent data obtained for samples collected after antiretroviral treatment (ART) initiation (Panel A), for samples with viral load<1,000 copies/mL (Panel B), and for samples with CD4 cell counts <350 cells/mm³ (Panel C). Panel D: The 4-peptide model described above was used to predict the duration of HIV infection in an independent sample set that included 72 samples from 32 participants in the GS Study (validation sample set, Table 1). Data were analyzed and plotted using the same methods used for Panels A-C. Red dots represent data obtained for samples with subtype D HIV. Correlation values are r=0.79 and r=0.64 for Panels A-C and D, respectively, under the assumption that data points are independent.

FIG. 6A-6C. Peptide engineering.

FIG. 6 illustrates antibody data for two representative parent peptides and their respective variant peptides generated by alanine scanning mutagenesis. High levels of antibody binding (z-scores>10) were observed in samples for all but one of the 57 participants for parent peptide A (98.2%) and for all 57 participants for parent peptide B. Panels A and B: These panels show heat maps of antibody binding for each set of peptides (the parent peptide and 54 variant peptides with triple alanine substitutions at different positions within the peptide); the position of the alanine substitution in each variant peptide is shown on y-axes. Antibody binding data are shown as a function of duration of HIV infection (x-axes). Panel C: The blue line shows antibody binding data (normalized read counts) for the parent peptide included in the analysis in panel B (parent peptide B) and selected variant peptides. Black lines show data for variant peptides with triple alanine substitutions at amino acids 12-17 and 19-21; the red line shows data for the variant peptide with the triple alanine substitution at amino acid 18.

Abbreviations: nrc: Normalized read count; mo: months; yr: years.

FIG. 7A-7B. Breadth of antibody reactivity for samples with low viral load and low CD4 cell count.

FIG. 7 illustrates the relationship between antibody breadth, HIV viral load, and CD4 cell count. The plots shown in this figure are the same as those shown in FIG. 2B, except that different data points are colored red. In Panel A, red dots indicate samples with viral loads <1,000 copies/mL (V_{L}<1,000). In Panel B, red dots indicate samples with CD4 cell counts <350 cells/mm³ (CD4<350).

Abbreviations: ART: antiretroviral treatment; V_{L}: viral load; mo: months; yr: years.

FIG. 8A-8B. Association of changes in antibody breadth and other factors.

FIG. 8 illustrates the relationship between the changes in antibody breadth between 9 months and 2 years after infection, time to initiation of antiretroviral therapy (ART), and other factors. Panel A: This plot shows univariate (pairwise) associations, reported as estimated Pearson correlation coefficients and respective p-values, between pairs of factors. Solid lines indicate correlations that were statistically significant after correction for multiple comparisons (p<0.05/15=0.0033). Panel B: The array shows histograms of data for factors evaluated for their association with time to ART initiation (diagonal). The array also shows scatter plots of the data (upper right) and summary statistics (lower left) for all pairwise comparisons. Summary statistics include the estimated Pearson correlations with 95% confidence intervals and the respective p-values. Units for variables are as follows: Age (years); viral load set point (log₁₀ copies/mL); baseline CD4 cell count (baseline CD4; cells/mm³); change in the antibody breadth between 9 months and 2 years after HIV infection; change in CD4 cell count between 9 months and 2 years after HIV infection (cells/mm³); time to ART (years).

FIG. 9. Association of peptide binding and duration of infection for the peptides selected based on the dynamics of antibody binding over the course of HIV infection.

FIG. 9 illustrates data from the four peptides in the 4-peptide model that was used to estimate the duration of HIV infection (peptides A-D); lines indicate longitudinal data for samples from each of the 57 study participants. Antibody binding (normalized read counts) is plotted as a function of duration of HIV infection. In each plot, the blue line is the locally-weighted regression curve (lowers smoother) for all participants, and the red line is the least squares regression line for all participants. P-values were calculated using generalized estimation equations to account for the dependency between measurements from multiple samples from each participant.

FIG. 10. Subtypes and strains of HIV represented by peptides in the VirScan library.

FIG. 10 illustrates the number of proteins and peptides in the VirScan peptide library corresponding to different HIV subtypes and strains.

FIG. 11. Peptides used to estimate the duration of HIV infection.

FIG. 11 illustrates information identifiers, amino acid sequences, protein location, and the position in the HIV genome for the peptides in the 4-peptide model that was used to estimate the duration of HIV infection. FIG. 11 discloses SEQ ID NOS 3, 22, 159, and 180, respectively, in order of appearance.

FIG. 12. Peptides that had antibody reactivity that was significantly associated with the duration of HIV infection.

FIG. 12 illustrates a list of 309 peptides for use in estimating HIV incidence and/or the duration of HIV infection. [Excel Spreadsheet]. The statistical association between antibody reactivity (measured as enrichment z-scores) and duration of infection was assessed for all HIV peptides in the VirScan library, using generalized estimation equations. The sign of the beta coefficient (positive or negative) indicates the observed direction of the association (positive or negative, respectively). All peptides exhibiting a p-value after adjustment for multiple comparisons using the Bonferroni method (“p.adj.Bonf”) of 0.05 or below are provided (309 peptides). Peptides included in the 4-peptide model are highlighted. FIG. 12 discloses SEQ ID NOS 1-309, respectively, in order of appearance.

FIG. 13. Samples used for analysis.

FIG. 13 illustrates characteristics of the participants who provided samples used in the analysis. The discovery sample set included 403 samples from 57 participants. The validation sample set included 72 samples from 32 participants who were not included in the discovery sample set.

Abbreviations: ART: antiretroviral therapy

### DETAILED DESCRIPTION OF THE INVENTION

The inventors used a massively-multiplexed antibody profiling system to analyze the fine specificity of the antibody response to HIV infection. This system is based on phage immunoprecipitation sequencing (PhIP-Seq) (7). Testing was performed by incubating samples with a bacteriophage library that expresses peptides encoded by oligonucleotides generated by high-throughput DNA synthesis. The abundance and specificity of antibodies in test samples were assessed by immunoprecipitating phage-antibody complexes and sequencing the DNA in the captured phage particles. The “VirScan” phage library includes >95,000 peptides that span the genomes of >200 viruses that infect humans (the human “virome”) (8). The inventors performed PhIP-Seq using the VirScan library to analyze HIV antibodies from individuals with known duration of HIV infection, ranging from <1 month to 8.7 years. This allowed them to examine dynamic changes in antibody diversity and the fine specificity of HIV antibodies from individuals with early to late stage infection, including individuals on antiretroviral therapy (ART) and individuals with advanced HIV disease.

HIV incidence was often determined by following cohorts of HIV-uninfected individuals and quantifying the rate of new HIV infections. HIV incidence can also be estimated using a cross-sectional study design, using laboratory assays to identify individuals who are likely to have recent HIV infection. Most serologic assays used for cross-sectional HIV incidence estimation measure general characteristics of the antibody response to HIV infection (e.g., antibody titer, antibody avidity) (9-11) which may be impacted by viral suppression, loss of CD4 T cells, and other factors (12-15). Unlike conventional methods the inventors used a VirScan assay to identify novel peptide biomarkers associated with the duration of HIV infection, and surprisingly demonstrated that peptide engineering can be used to enhance the properties of peptides for discriminating between early and late-stage infection. This information could be used to develop improved methods for estimating HIV incidence from cross-sectional surveys, for surveillance of the HIV/AIDS epidemic, and evaluating the impact of interventions for HIV prevention in clinical trials.

Antibody reactivity to HIV peptides.

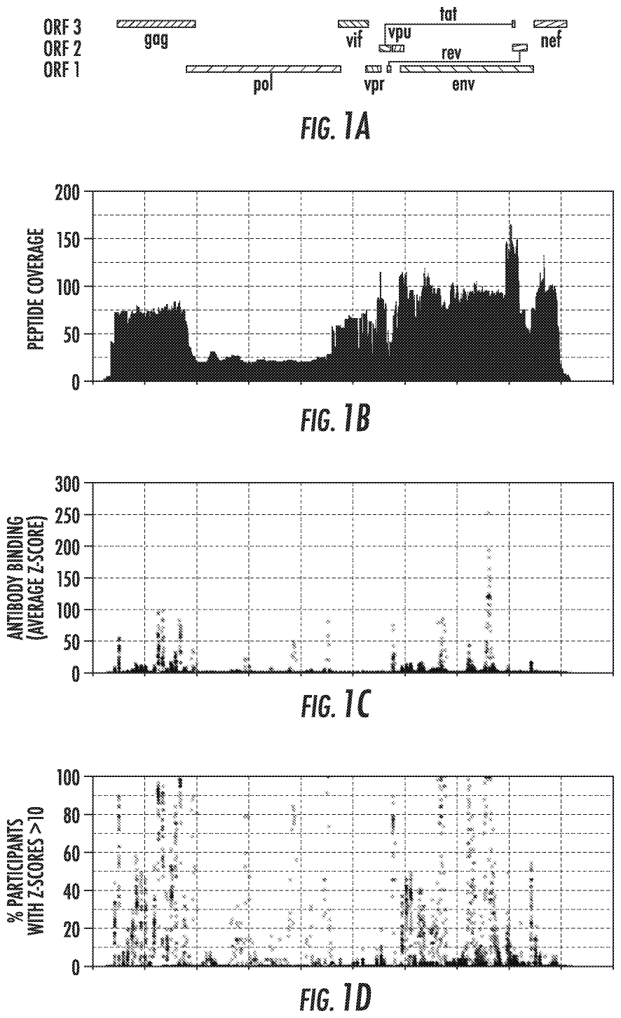

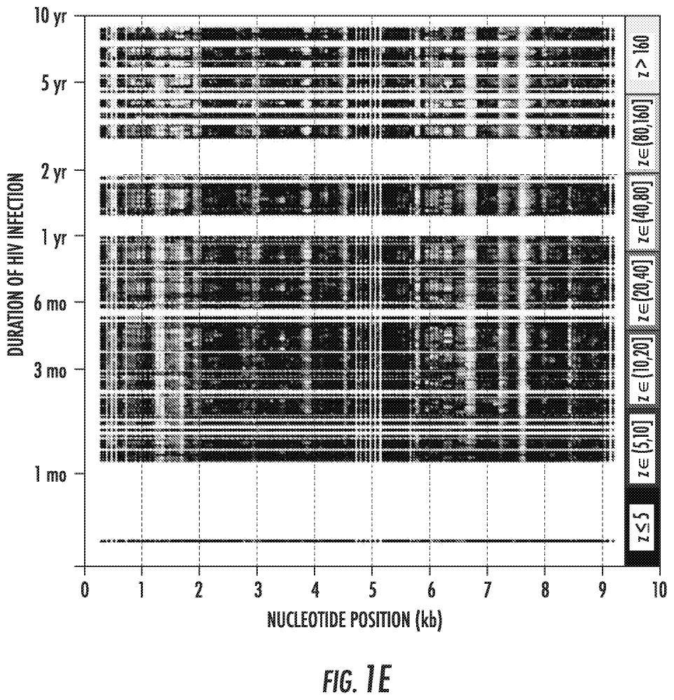

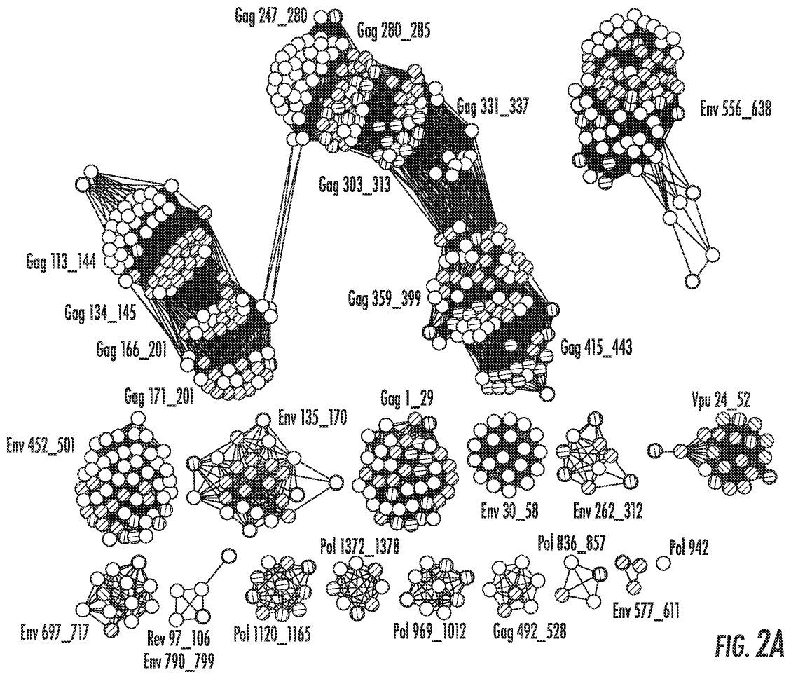

We used the VirScan assay to characterize anti-HIV antibodies in 403 plasma samples from 57 women with subtype C HIV infection (FIG. 13). The time from seroconversion to sample collection ranged from 14 days to 8.7 years. The density of peptides in the library varied across the open reading frames for HIV proteins (the HIV proteome, FIGS. 1A and 1B). The level and frequency of antibody binding were highly variable (FIGS. 1C and 1D); the strongest and most frequent antibody binding was observed for peptides in the gag and env regions. Some peptides were consistently targeted over the course of the infection; in contrast, the level and frequency of antibody binding to other peptides increased or decreased over the course of HIV infection (FIG. 1E).

Breadth of antibody reactivity.

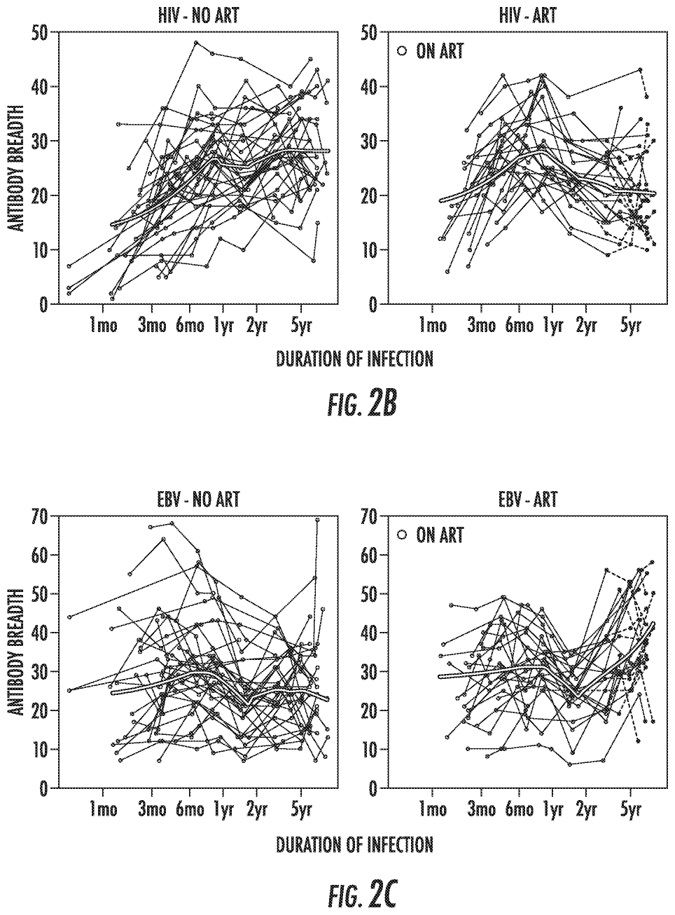

The inventors next analyzed the diversity of each individual's antibody response to HIV over time. Network graphs were used to determine antibody breadth at each time point; antibody breadth was defined as the number of non-overlapping peptides with high levels of antibody binding. FIG. 2A shows the network graph for peptides that reacted with antibodies from a representative study sample (one immunoprecipitation reaction). This analysis identified 45 non-overlapping peptides; these peptides were located in the gag, pol, env, vpu and rev regions. The inventors next analyzed the change in antibody breadth over the course of HIV infection. Since ART was known to influence HIV antibody production, the inventors compared data from participants who did vs. did not start ART during the GS Study (FIG. 2B). ART also serves as a surrogate for disease progression; in the GS Study, ART was recommended when the CD4 cell count fell below 250 cells/mm³. Overall, 32 participants started ART during the GS Study.

In both groups (with and without ART initiation), antibody breadth increased during the first 6 months of infection. In the group that did not start ART, a relatively stable value for antibody breadth (termed “antibody breadth set point”) was established in most individuals approximately nine months to one year after infection; the antibody breadth set point varied considerably among study participants. In contrast, in the group that ultimately started ART, a decline in antibody breadth was observed approximately one year after infection. After participants started ART, antibody breadth appeared to stabilize at levels similar to those seen in early HIV infection. The decline in antibody breadth prior to ART initiation did not appear to be related to HIV viral load or CD4 cell count (FIG. 7).

The inventors next evaluated the relationship between HIV infection and the antibody response to a different, chronic infection that was expected to have a high prevalence in the study setting (EBV) (FIG. 2C). Data used to calculate the breadth of the antibody response to EBV infection were obtained from the same VirScan data sets used for HIV analysis (FIG. 2C). In most participants, EBV antibody breadth was relatively stable in the first 6 months of HIV infection, and then declined. EBV antibody breadth then appeared to stabilize in participants who did not start ART for HIV infection. In contrast, in most participants who started ART, EBV antibody breadth increased after ART initiation, often reaching levels that surpassed those observed early in HIV infection.

Factors associated with changes in antibody breadth over time.

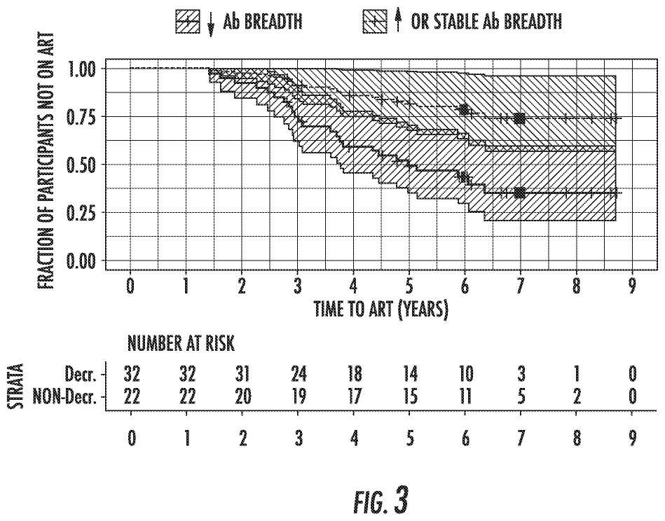

To explore the relationship between the decline in HIV antibody breadth and subsequent ART initiation, the inventors calculated the rate of change of antibody breadth over the period ˜9 months to ˜2 years after HIV infection (based on sample availability); none of the participants included in the analysis were on ART during this time window. For this time-to-event analysis (the outcome being time to ART initiation), participants were divided into two groups: those with declining breadth and those with stable or increasing breadth. The inventors found that participants who had stable or increasing antibody breadth ˜9 months to ˜2 years after infection were less likely to start ART earlier in infection (log-rank test p=0.009, hazards ratio: 0.29, 95% CI: 0.11, 0.78, p=0.014, FIG. 3). The average time between the study visits used to evaluate the change in antibody breadth (˜9 months and ˜2 years after infection) was similar in the two groups (p=0.28), so this was not likely to have biased the analysis.

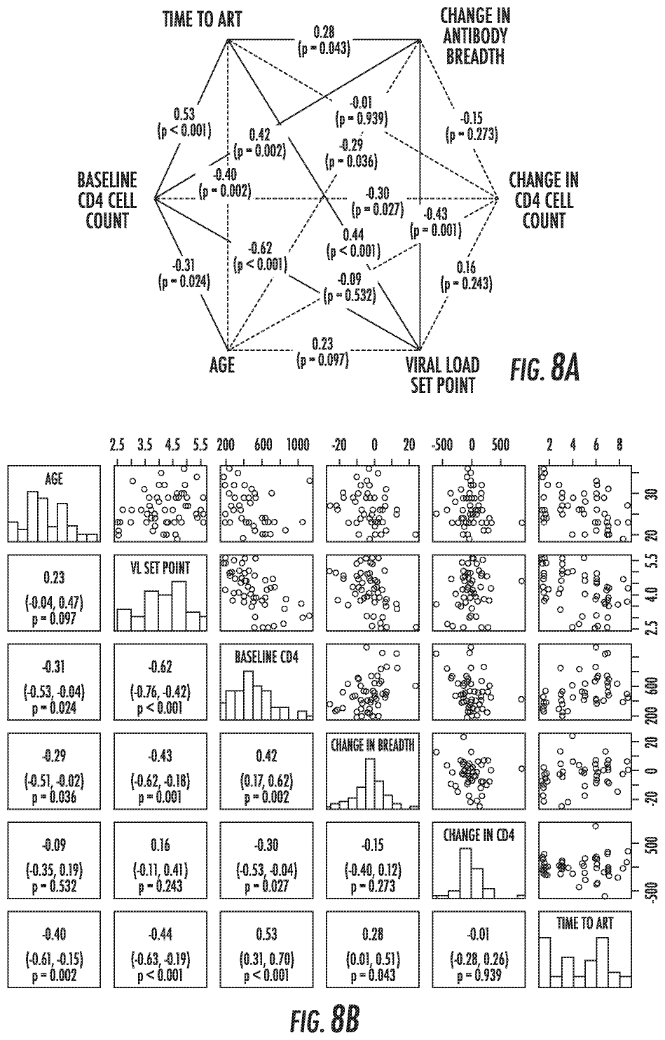

The inventors next evaluated the relationship between the rate of decline in antibody breadth and other factors, including age at infection, baseline CD4 cell count, rate of decline in CD4 cell count, and viral load set point (FIG. 8). A faster decline in antibody breadth was strongly associated with lower baseline CD4 cell count (R=0.42, 95% CI: 0.17, 0.62; p=0.002) and higher viral load set point (R=−0.43, 95% CI: −0.62, −0.18; p=0.001), and was also associated with earlier ART initiation (R=0.28, 95% CI: 0.01, 0.51; p=0.043).

Dynamic Changes in Antibody Binding

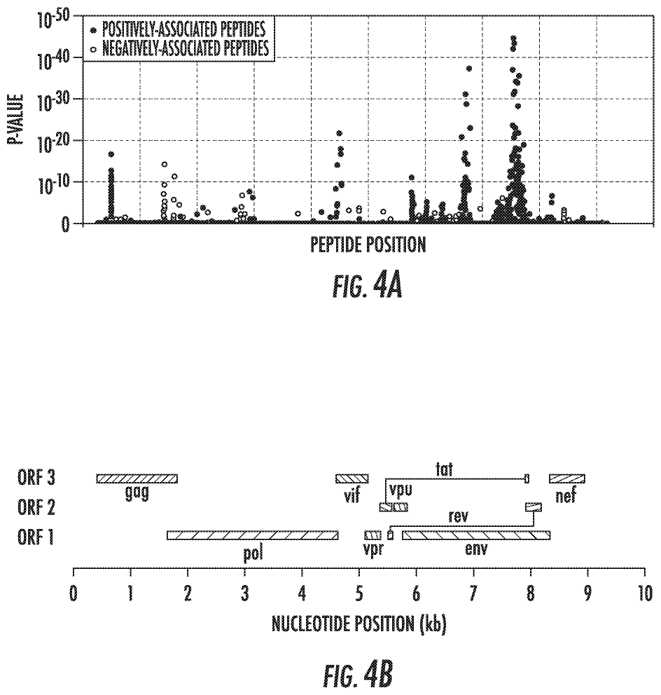

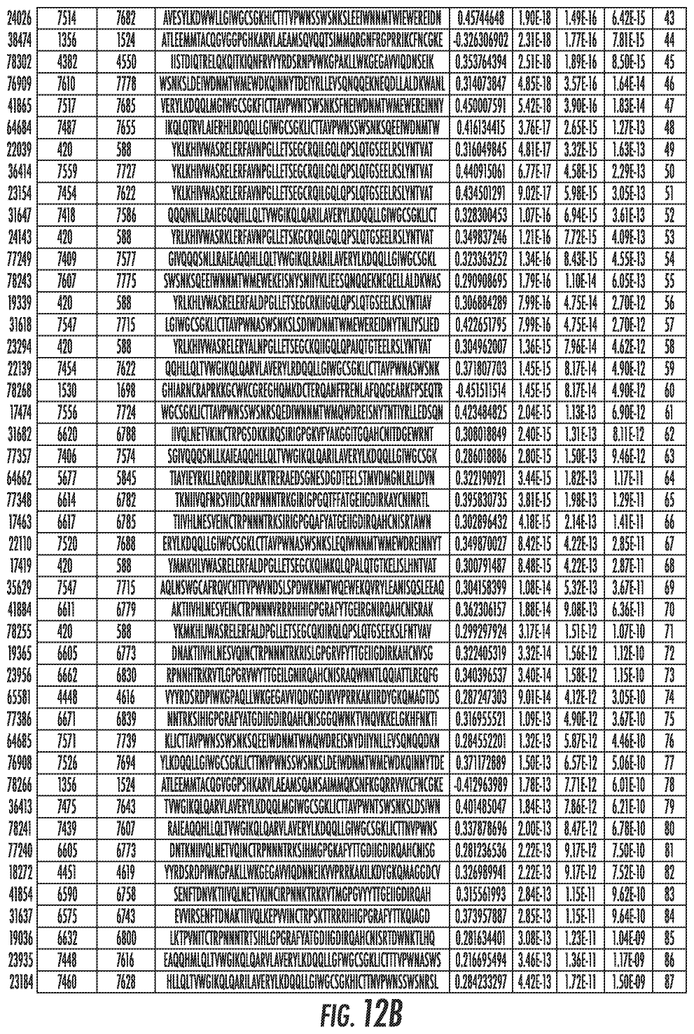

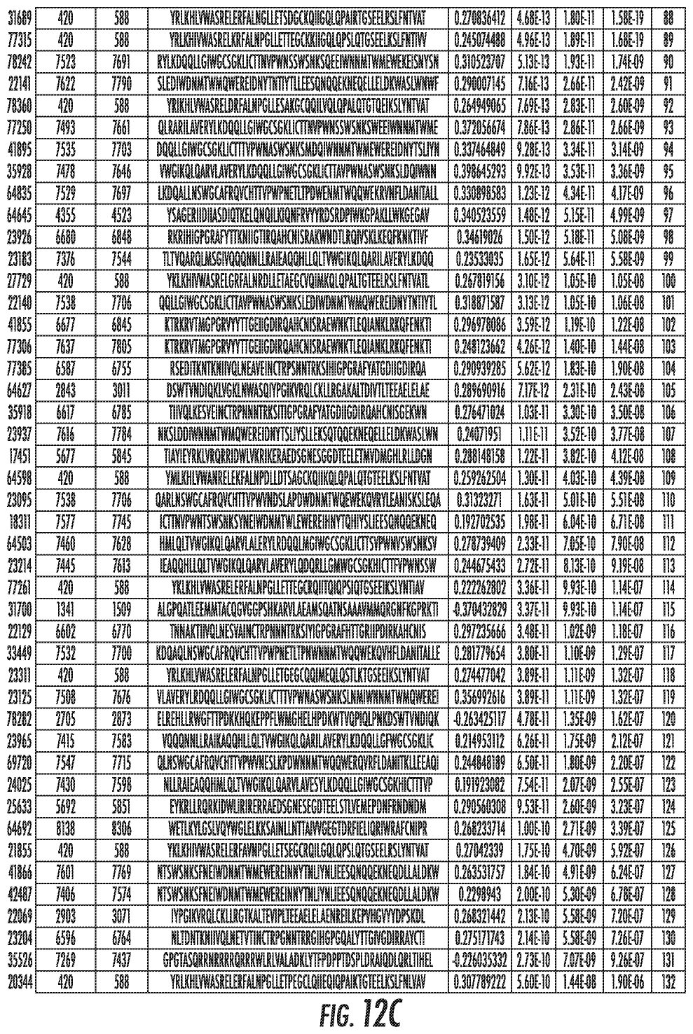

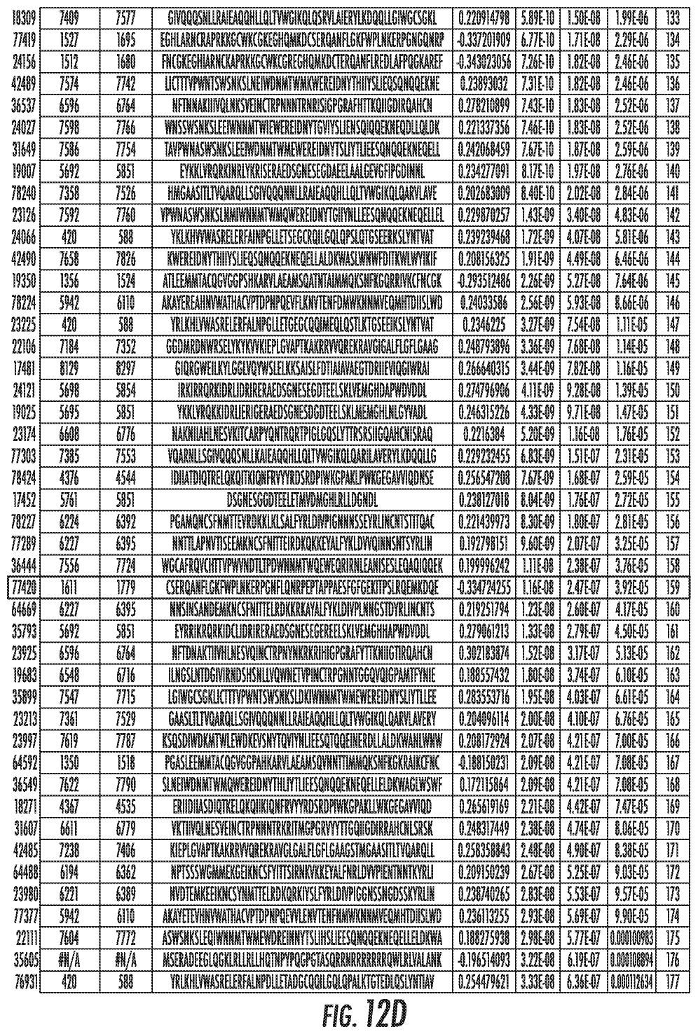

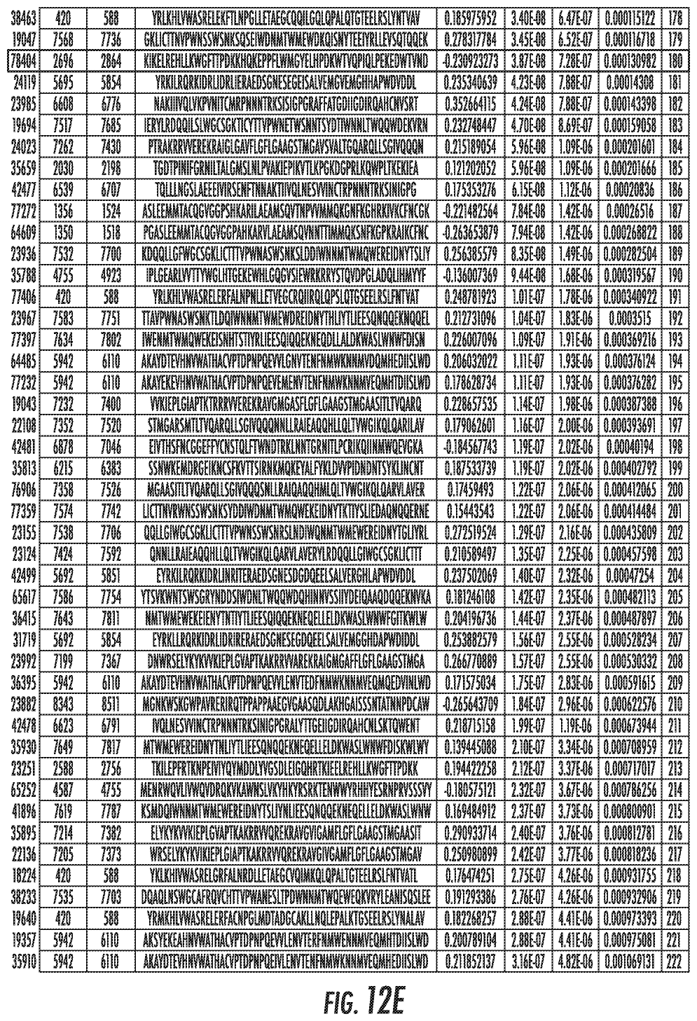

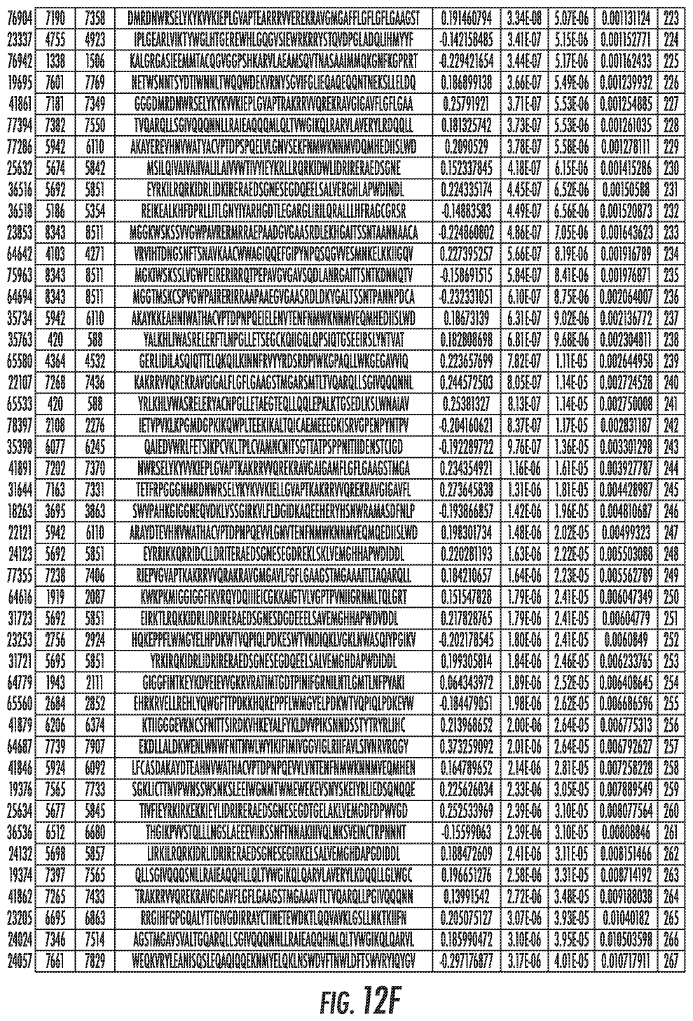

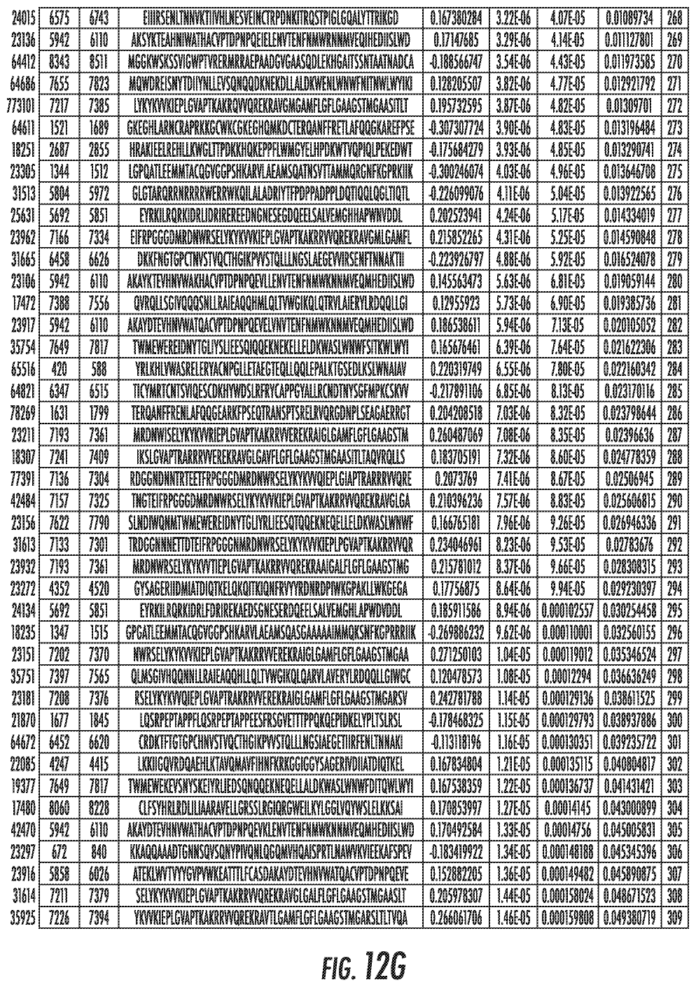

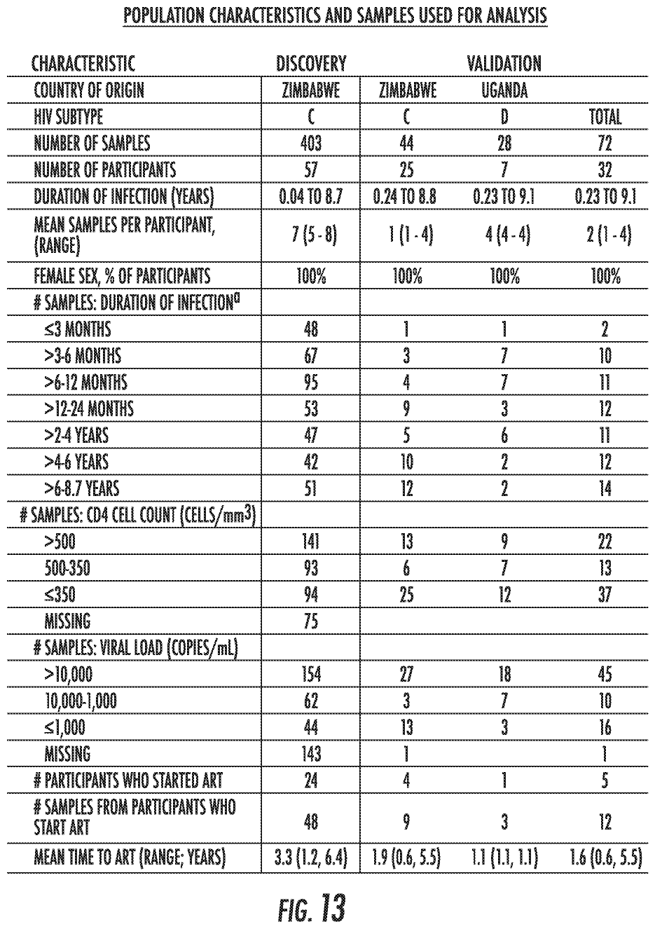

The inventors next explored the relationship between HIV antibody specificity and the duration of HIV infection. First, the inventors used a linear model to quantify the association between antibody binding and the duration of infection for the 3,384 HIV peptides in the VirScan library. This analysis was performed using all 403 samples in the discovery sample set. The model identified 309 peptides that had a significant association between these two factors (p-value<0.05 after adjusting for multiple comparisons using the Bonferroni method, FIG. 4A and FIG. 12); 266 peptides had increasing antibody binding over time (positive association) and 43 peptides had decreasing antibody binding over time (negative association). The position of peaks representing increased vs. decreased antibody binding were observed at different positions in the HIV genome. Peptides that had a strong positive association with duration of infection tended to cluster in the N-terminal gag region, the C-terminal pol region, and defined domains within the env region. In contrast, peptides that had a strong negative association with duration of infection clustered in the C-terminal gag region, and the middle of the pol region, with others scattered across the env region or located in non-structural (accessory) proteins, such as nef.

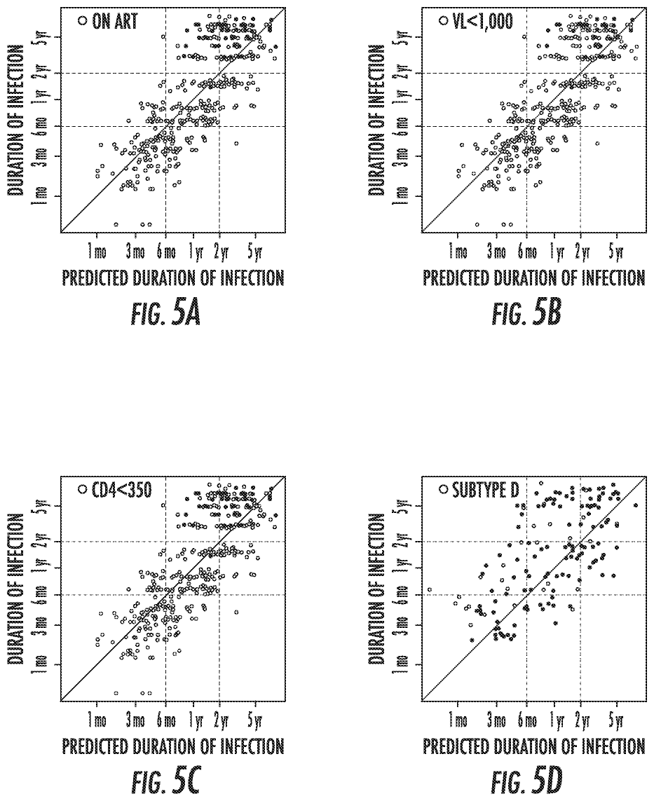

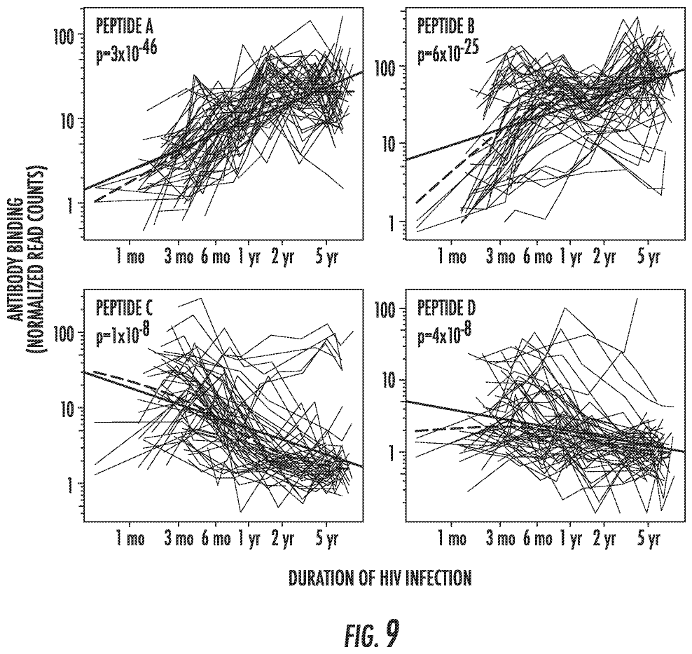

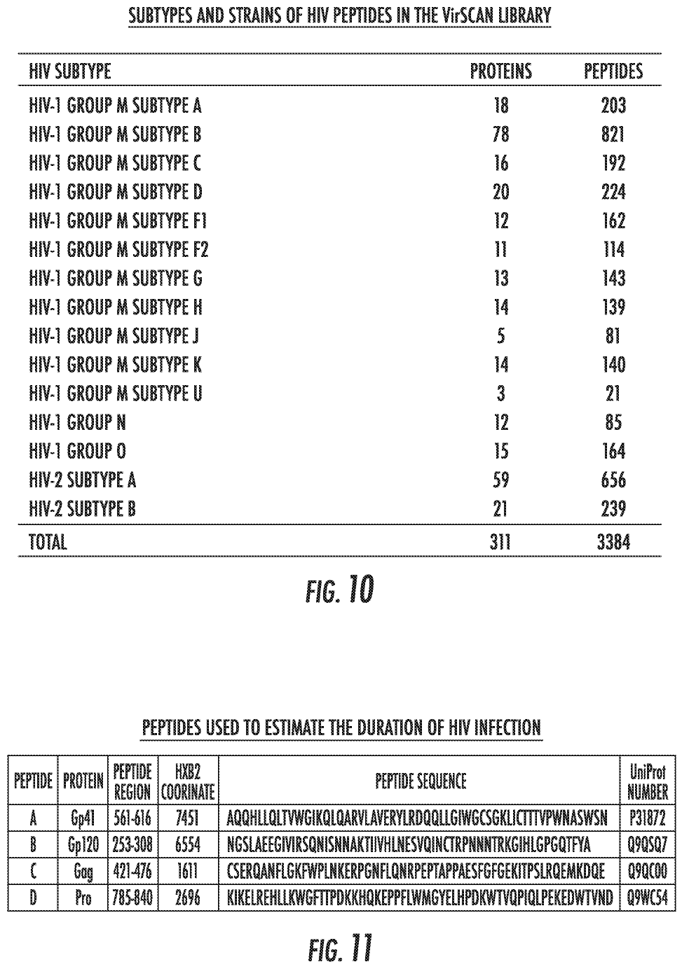

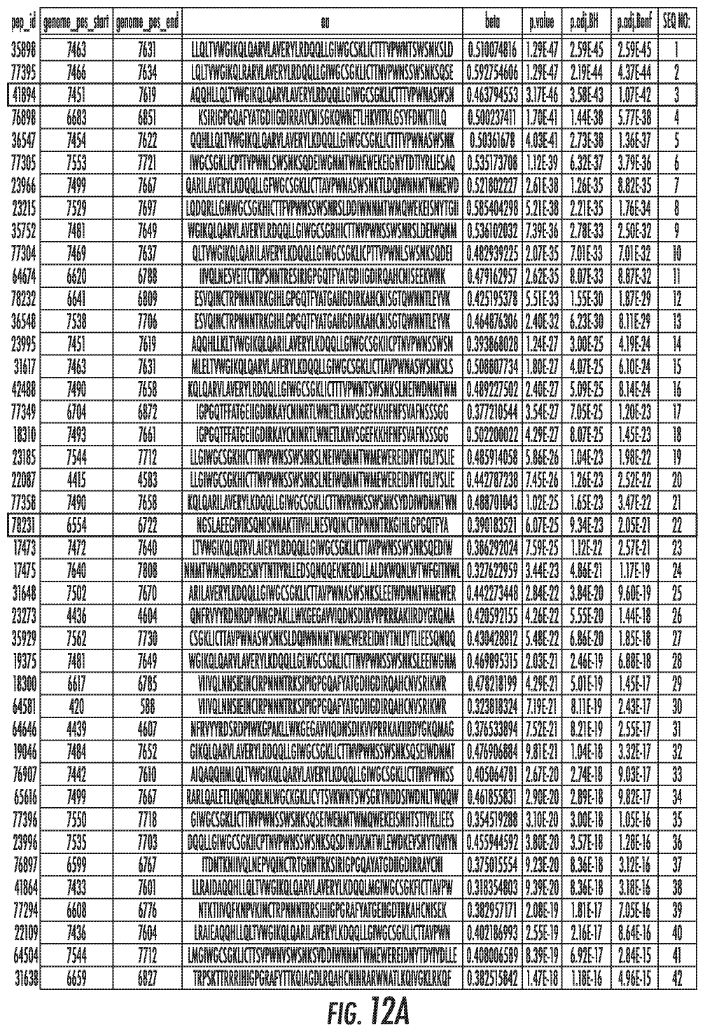

The inventors then selected the four peptides that had the strongest independent association between antibody binding and the duration of HIV infection (FIG. 9 and FIG. 11). This included two peptides that had increased antibody binding over time (one in gp41; one in gp120), and two peptides that had decreased antibody binding over time (one in gag; one in pol). Antibody binding measures from each of the four peptides were combined in a simple linear model to generate a single, unweighted, 4-peptide composite measure. The duration of infection predicted by this model was highly correlated with the observed (true) duration of infection (GEE p<1×10⁻¹⁰⁰; FIG. 5A). Importantly, the predictive value of the 4-peptide composite measure did not appear to be impacted by ART initiation, low viral load, or low CD4 cell count (FIG. 5A-C).

The inventors next evaluated the performance of the 4-peptide model using an independent validation sample set (FIG. 13). This set consisted of samples from individuals in the GS Study who were not included in the discovery set that was used to identify the model peptides. This sample set also included “challenge samples” that have characteristics known to complicate cross-sectional HIV incidence estimation using other serologic assays: 28 (38.9%) of the samples were HIV subtype D; 37 (51.4%) had CD4 cell counts <350 cells/mm³; 16 (22.2%) had viral loads <1,000 copies/mL, and 12 (16.7%) were from individuals on ART. The duration of infection predicted by the 4-peptide model was also correlated with the observed (true) duration of infection using this independent sample set (GEE p<3×10⁻³⁶; FIG. 5D). The predictive value of the 4-peptide composite measure did not appear to be not impacted by HIV subtype (subtype C vs. D; FIG. 5D).

Epitope Engineering

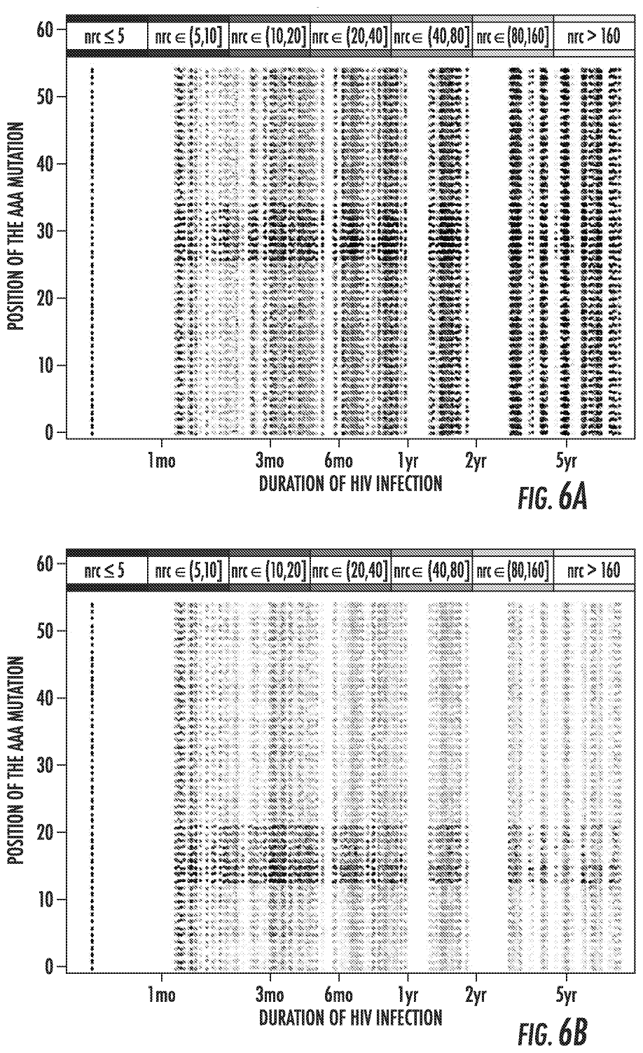

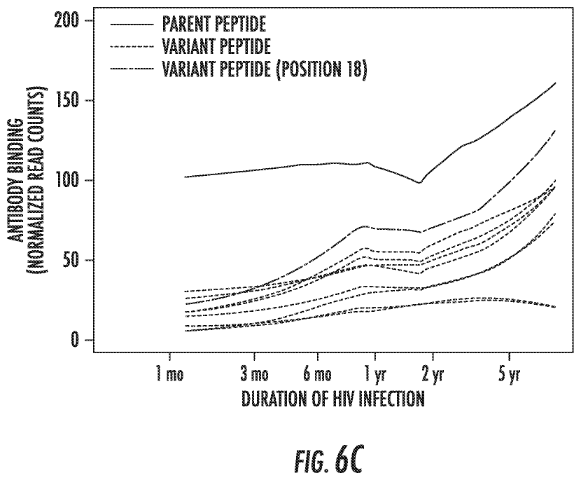

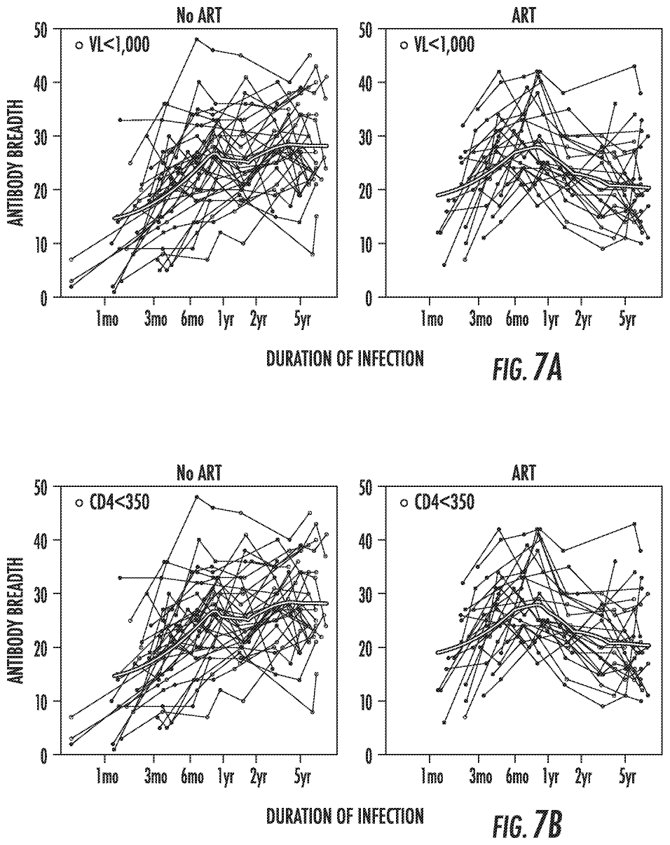

Next, the inventors explored whether peptide epitopes could be modified to improve the association between antibody binding and the duration of HIV infection. The inventors first selected 11 non-overlapping peptides that were targeted by the majority of HIV-infected individuals (“public epitope peptides”). The inventors then generated variant peptides by substituting each set of three consecutive amino acids with alanine residues. FIG. 6 shows the impact of alanine substitutions on antibody binding for two of the 11 parent peptides; these peptides were targeted by >98% of the study participants. In the first case (parent peptide A), antibody binding to the parent peptide and most of the variant peptides decreased with increasing duration of infection (FIG. 6A). Alanine substitutions at amino acid positions 26-34 appeared to disrupt antibody binding at all time points. In the second case (parent peptide B), antibody binding to the parent peptide and most of the variant peptides increased with increasing duration of infection (FIG. 6B). In this case, alanine substitutions at amino acid positions 13-21 preferentially disrupted antibody binding early in infection. FIG. 6C shows the level of antibody binding as a function of duration of infection for parent peptide B and variant peptides that had alanine substitutions in the region most impacted by mutagenesis (9 peptides, with substitutions at positions 13-21). Over the course of HIV infection, antibody binding to the parent peptide increased by 57%; in contrast, antibody binding to one of the variant peptides increased by approximately 479% over the same time period. These data provide proof-of-principle that epitope engineering can be used to improve the capacity of peptides to serve as quantitative biomarkers of disease processes, such as the duration of HIV infection.

The present invention provides the most comprehensive analysis of HIV antibody specificities to date, including their characterization from early to late stage infection. The inventors found that changes in antibody diversity early in infection were associated with differences in clinical outcome (measured as time to ART initiation). This study also provides proof-of-principle that an “HIV serosignature”, reactivity to a panel of HIV peptides, is useful for cross-sectional HIV incidence estimation.

The inventors used a novel definition of “antibody breadth” to quantify HIV antibody diversity, and found that this measure reaches a plateau (“antibody breadth set point”) early in infection. In the GS study cohort, a decline in antibody breadth between 9 months and 2 years after infection was associated with a shorter time to ART initiation, which was prompted in the GS Study cohort by a decline in CD4 cell count to <250 cells/mm³. The decline in antibody breadth among those who subsequently started ART likely reflected declining B cell support due to loss of T helper cells. HIV antibody breadth appeared to stabilize at a low level after ART initiation. In contrast, the breadth of the EBV antibody response increased sharply after ART initiation, which may have reflected immune reconstitution.

Previous studies have identified several factors associated with HIV disease progression, including virologic factors [e.g., HIV viral load, replication capacity, and subtype], immunologic factors [e.g., inversion of the CD4/CD8 ratio, polyclonality of the anti-HIV T cell response, degree of early immune activation] and host factors [e.g., human leukocyte antigen (HLA) type B57, CCR5 delta 32 mutations]. It is not clear if the decline in antibody breadth that we observed caused disease progression leading to ART initiation, or if it was a surrogate for other changes, such as a decline in T cell number or function. If the decline in antibody breadth has a causative role in disease progression, then use of therapeutic vaccines to boost antibody diversity may in theory provide clinical benefit.

Generalized antibody responses to HIV infection, such as antibody titer and avidity, tend to plateau approximately one year after HIV infection. These characteristics of the antibody response are impacted by a variety of factors, including natural and drug-induced viral suppression, disease progression, and HIV subtype. Previous studies evaluating the banding pattern in Western blots demonstrate that HIV antibody specificity evolves early in infection. Recent studies have explored whether assays that include a small number of protein or peptide targets could be used to identify recent HIV infections. Using the VirScan assay to analyze 403 plasma samples, the inventors were able to quantify antibody binding to >3,300 HIV peptides from early to late-stage HIV infection. These data were used to generate a simple, unweighted, 4-peptide model that predicted duration of HIV infection. The peptides included in this prototype model were from four different HIV proteins (gp41, gp120, gag and pol). Two of these peptides had increasing antibody reactivity over time, and two had decreasing antibody reactivity over time. It is noteworthy that the gp41 peptide, which showed the strongest association with duration of infection, included a sequence shared by the HIV subtype B target peptide in the Limiting Antigen Avidity (LAg) assay that is in wide use for cross-sectional HIV incidence estimation. Our analysis also demonstrated that epitope engineering can be used to enhance the capacity of individual peptides to discriminate between early and late HIV infection.

Data obtained with the 4-peptide model described above demonstrates that the VirScan assay can be used to identify peptides for applications such as cross-sectional HIV incidence estimation. The inventors are currently investigating more sophisticated statistical and machine-learning models to identify peptide combinations with greater accuracy for predicting the duration of HIV infection, and are generating larger data sets for model building and assessment. We are also exploring whether alternate serosignatures provide more accurate prediction of the duration of infection among people with longer term infections. On-going studies will also provide more information about the possible impact of ART, viral load, and CD4 cell count on antibody binding profiles. Considerable work will be needed to translate findings from this study into a laboratory test that can be used for improved cross-sectional HIV incidence testing. For example, peptides of interest could be incorporated into high-resolution, quantitative, multi-peptide enzyme immunoassays (EIAs) for high-throughput testing. Antibody binding data obtained from the EIA testing platform could then be used to compare the performance of serosignatures for HIV incidence estimation that include different sets of peptides, weighting for individual peptides, and different cut-offs for antibody binding to each peptide in the model. In previous work, we have used this approach to identify multi-assay algorithms that provide accurate cross-sectional HIV incidence estimates.

The VirScan assay has several unique advantages over alternative multiplex serological assays for peptide discovery. These include: quantitative assessment of antibody binding to peptides that span all open reading frames in the HIV genome, including both structural and regulatory proteins; representation of a wide range of HIV subtypes and strains, including groups M, N, and O and HIV-2; and fine resolution for epitope identification, which can be further refined with alanine scanning mutagenesis. The assay also provides information about antibody binding to >200 other human viruses. In this report, data from other viral peptides were used to normalize peptide binding measures, and allowed us to compare the impact of ART on the antibody response to a prevalent non-HIV viral infection (EBV). Data from the same assay runs could be used to examine the evolution and fine specificity of antibodies to other viruses, and the impact of viral co-infections on the anti-HIV antibody response. Future studies could also explore use of the VirScan assay to identify serosignatures for estimating incidence of other viral infections, such as hepatitis C virus. Finally, future phage libraries composed of additional protein products, such as those from the gut microbiome, may be used to explore the impact of immune system pre-conditioning on the response to HIV infection.

This present invention reveals novel features of the humoral response to HIV infection, and demonstrates the utility of the VirScan assay for identifying peptide biomarkers for applications such as cross-sectional HIV incidence estimation. This technology could also be used to evaluate serologic responses to other infectious diseases, as well as the impact of viral co-infections on immune responses. This may improve understanding of the complex relationships between viral infections and the immune responses that they elicit.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

Samples Used for Analysis

Plasma samples were obtained from the GS Study (Uganda and Zimbabwe; 2001-2009), which evaluated the relationship between hormonal contraceptive use, genital shedding of HIV, and HIV disease progression among women with known dates of HIV seroconversion (18). ART was recommended for study participants with CD4 cell counts below 250 cells/mm³, consistent with local treatment guidelines at the time the GS Study was performed. Data for CD4 cell count and viral load were collected in the GS Study (18); data on the timing of ART initiation was obtained by review of clinic records.

The inventors analyzed samples from participants who acquired HIV infection, where the maximum time between collection of the last HIV-negative sample and the first HIV-positive sample was four months. For each individual, the estimated date of infection was defined as either the midpoint between visits with the last negative HIV antibody test and the first positive HIV antibody test, or fifteen days before documentation of acute infection (HIV RNA positive/HIV antibody negative status). Two sets of samples were analyzed in this report: a discovery sample set and a validation sample set (FIG. 13). The discovery sample set included participants who had at least one year of follow-up after seroconversion, with samples collected at three or more study visits during that period. The independent validation sample set included samples from participants from the GS Study who were not included in the discovery sample set. HIV subtype assignments were based on phylogenetic analysis of the HIV env C2V3 region (19). All of the samples in the discovery sample set were HIV subtype C; the validation sample set also included “challenge” samples with HIV subtype D, which are often misclassified using currently available serologic HIV incidence assays (20, 21).

Phage Library Used for Analysis

The VirScan library includes 3,384 HIV peptides spanning all HIV proteins (8). The protein sequences used to design peptide tiles were selected from the UniProtKB database, balancing sequence diversity and library size (8). The peptides are 56-amino acids long with 28-amino acid overlaps and represent diverse HIV subtypes and strains (FIG. 10). In this study, the VirScan library was augmented with a public epitope library that included peptides previously found to be targeted by a high proportion of HIV-infected individuals (8). Eleven “parent” peptides in the public epitope library were modified by introducing triple alanine substitutions centered at each amino acid position; the resulting public epitope library included 594 genetically-engineered variant HIV peptides. Silent nucleotide substitutions were encoded in the first 50 nucleotides of each DNA tile, so that variant peptides could be uniquely identified using 50-nucleotide single-end Illumina sequencing.

The VirScan library also includes 2,263 Epstein Barr virus (EBV) peptides, 718 Ebola virus peptides, and 518 rabies virus peptides; the public epitope library includes an additional 227 Ebola virus peptides. In this report, EBV data were used to evaluate the impact of antiretroviral therapy for HIV infection on the breadth of the anti-EBV antibody response. Ebola and rabies virus data were used to normalize antibody binding data to account for differences in sequencing depth between samples.

Phage Immunoprecipitation and DNA Sequencing

Detailed procedures for the VirScan assay were described previously (8, 22). In this study, the concentration of IgG in plasma samples was determined using an in-house enzyme-linked immunosorbent assay (capture and detection antibodies 2040-01 and 2042-05, respectively Southern Biotech, Birmingham, Ala.). Approximately 2 μg of IgG from each sample were added to the combined T7 bacteriophage VirScan and public epitope libraries (1×10⁵ plaque forming units for each phage clone in each library), diluted in phosphate-buffered saline to a final reaction volume of 1 mL in a deep 96-well plate, and incubated overnight at 4° C. Eight mock immunoprecipitation reactions (no plasma) were included on each plate; these reactions served as negative controls for data normalization. After rotating the plates overnight at 4° C., 20 μL of protein A-coated magnetic beads and 20 μL of protein G-coated beads (catalog numbers 10002D and 10004D, Invitrogen, Carlsbad, Calif.) were added to each reaction; the plates were rotated for another 4 hours at 4° C. Immunoprecipitation reactions were processed using the Agilent Bravo liquid handling system (Agilent Technologies, Santa Clara, Calif.). Beads were washed twice with Tris-buffered saline (50 mM Tris-HCl with 150 mM NaCl, pH 7.5) containing 0.1% NP-40 and then resuspended in 20 μL of a polymerase chain reaction (PCR) mix containing Herculase II Polymerase (catalog number 600679, Agilent Technologies). After 20 cycles of PCR, 2 μL of the PCR products was added to a second 20-cycle PCR reaction, which added sample-specific barcodes and P5/P7 Illumina sequencing adapters to the amplified DNA. DNA sequencing of the pooled PCR products was performed using an Illumina HiSeq 2500 instrument (Illumina, San Diego, Calif.) in rapid mode (50 cycles, single end reads).

Analysis of DNA Sequencing Data

Fastq files from DNA sequencing were demultiplexed using exact matching of 8-nucleotide sample-specific i5 and i7 DNA barcodes (Illumina). For each sample, a read count (the number of times each sequence was detected) was obtained for each peptide using Bowtie alignment (23), without allowing any mismatches. The level of antibody-dependent enrichment of each peptide in each sample was determined by comparing the read count for the sample to the read counts obtained for 40 mock immunoprecipitation reactions (8 mock reactions per plate). Two different measures were used to quantify the degree of antibody binding: “z-scores” were used to reduce false positivity in cases of low sequencing depth (this approach was used to generate data for FIG. 1 and for calculation of antibody breath); “relative fold-change” was used to normalize data for highly-enriched peptides (this approach was used to generate data for FIGS. 4-6 and FIG. 9). Z-scores were calculated by subtracting the expected normalized read count (determined by regression against the mock immunoprecipitation reactions) from the observed normalized read count; the resulting value was then divided by an estimate of the standard deviation of the normalized read counts, based on the mock immunoprecipitation reactions (24). Relative fold change values were determined as follows. Read counts were log₁₀ transformed prior to analysis. First, read count data for Ebola virus and rabies virus was trimmed by removing outlier values (the lowest 5% and highest 5%). The log₁₀ transformed read count for each HIV peptide (after adding one read count) was then normalized to the average read count for all Ebola virus and rabies virus peptides of the respective sample. To generate a fold change value for each HIV peptide, the normalized value of the peptide was divided by the average of the normalized values for the same peptide observed across the mock immunoprecipitation reactions that were run on the same plate.

Determination of Antibody Breadth

The term, “antibody breadth”, was used to indicate the number of unique non-overlapping epitopes that had high levels of antibody binding (z-scores>10). Antibody breadth was determined for HIV and EBV peptides using network graphs as follows. The amino acid sequences of all peptides in the VirScan library (HIV or EBV) were first analyzed to identify sequence overlaps (linkages, defined as two peptides sharing an identical sequence at least 7 amino acids long). The linkages were used to construct an undirected network graph, where each node represented a peptide with high-level antibody binding, and each linkage between two nodes represented a sequence overlap between the two peptides. The number of linkages for each peptide defined its degree of connectivity. Peptides were then removed from the graph one at a time using the following approach. At each iteration, the peptide(s) with maximum connectivity was removed, and the degree of connectivity was recalculated for each of the remaining peptides. If multiple linked peptides had equivalent connectivity, the peptide with the lowest z-score was removed first. This process was repeated until the only remaining structures in the network were simple paths and cycles. For cycles (simple paths without end peptides), the peptide with the lowest z-score was removed first; this resulted in a simple path. Peptides were iteratively removed from simple paths in order to retain the greatest number of unlinked peptides. The number of remaining unlinked peptides was defined as the antibody breadth (25).

Rate of Change in Antibody Breadth

For each participant, we estimated the rate of change in antibody breadth over the time period from 9 months to 2 years after HIV infection. This was calculated by determining the difference in antibody breadth for samples collected closest to time points 9 months and 2 years after HIV infection, and dividing this value by the length of time between the two visits. The rate of change in CD4 cell count was derived in the same way, using samples that had associated CD4 cell count data. The relationship between the rate of change in antibody breadth (and other factors) with time to ART initiation was determined using Cox proportional hazards models. The following factors were included in the analysis: age at seroconversion, CD4 cell count at the first visit after seroconversion, viral load set point, the rate of change in CD4 cell count, and time between HIV seroconversion and ART initiation. Viral load set point was defined as the median log₁₀ viral load, excluding viral load results from the first HIV-positive visit, the visit prior to ART initiation, and any visits after ART initiation. Pearson correlation coefficients and their respective p-values and 95% confidence intervals were used to describe the relationships between the factors analyzed. We also compared the time to ART initiation among individuals who experienced a decline in antibody breath between 9 months and 2 years, and those who had stable or increasing antibody breadth in this period. Statistical significance between the breadth measures and time to ART initiation was assessed using a non-parametric log-rank test and the semi-parametric Cox proportional-hazards model with a dichotomized variable for change in breadth rate (decreasing vs. stable/increasing). Individuals who did not initiate ART were treated as right-censored. Survival curves were plotted based on the resulting hazard functions for the two groups.

Identification of peptides for estimating duration of HIV infection.

The observed duration of infection (log₁₀ transformed) was regressed on each of the normalized read count for each peptide, and the peptide with the strongest association was selected. To select additional peptides with independent information about duration of infection, we correlated the “residuals” (i.e., the differences between the observed and fitted values) from the above linear model against each of the remaining peptides, selected the peptide with the strongest association, and repeated this step twice more to generate a list of four peptides. Two of the four peptides had increased antibody binding over time since infection (positively associated with duration of infection), and two had decreasing antibody binding over time (negatively associated with duration of infection). A simple predictor for duration of infection was calculated as the sum of the normalized read counts for the positively-associated peptides, minus the sum of the normalized read counts for the negatively-associated peptides; read counts were log transformed for this analysis. For the analysis of predicted duration of infection, generalized estimating equations (GEE) were used to account for auto-regressive correlation structure of samples from the same individual.

### REFERENCES

All references, including publications, patent applications, and patents, cited herein are hereby incorporated by reference to the same extent as if each reference were individually and specifically indicated to be incorporated by reference and were set forth in its entirety herein.
1. S. K. Wendel et al., Effect of natural and ARV-induced viral suppression and viral breakthrough on anti-HIV antibody proportion and avidity in patients with HIV-1 subtype B infection. PLoS One 8, e55525 (2013).
2. E. W. Fiebig et al., Dynamics of HIV viremia and antibody seroconversion in plasma donors: implications for diagnosis and staging of primary HIV infection. AIDS 17, 1871-1879 (2003).
3. Y. Geiss, U. Dietrich, Catch Me If You Can—The Race Between HIV and Neutralizing Antibodies. AIDS Rev 17, 107-113 (2015).
4. E. Y. Dotsey et al., A High Throughput Protein Microarray Approach to Classify HIV Monoclonal Antibodies and Variant Antigens. PLoS One 10, e0125581 (2015).
5. K. A. Curtis et al., Development and characterization of a bead-based, multiplex assay for estimation of recent HIV type 1 infection. AIDS Res Hum Retroviruses 28, 188-197 (2012).
6. S. Delhalle, J. C. Schmit, A. Chevigne, Phages and HIV-1: from display to interplay. Int J Mol Sci 13, 4727-4794 (2012).
7. H. B. Larman et al., Autoantigen discovery with a synthetic human peptidome. Nat Biotechnol 29, 535-541 (2011).
8. G. J. Xu et al., Viral immunology. Comprehensive serological profiling of human populations using a synthetic human virome. Science 348, aaa0698 (2015).
9. G. Murphy, J. V. Parry, Assays for the detection of recent infections with human immunodeficiency virus type 1. Euro Surveill 13, (2008).
10. R. Guy et al., Accuracy of serological assays for detection of recent infection with HIV and estimation of population incidence: a systematic review. Lancet Infect Dis 9, 747-759 (2009).
11. M. P. Busch et al., Beyond detuning: 10 years of progress and new challenges in the development and application of assays for HIV incidence estimation. AIDS 24, 2763-2771 (2010).
12. O. Laeyendecker et al., Factors associated with incorrect identification of recent HIV infection using the BED capture immunoassay. AIDS Res Hum Retroviruses 28, 816-822 (2012).
13. O. Laeyendecker et al., Specificity of four laboratory approaches for cross-sectional HIV incidence determination: analysis of samples from adults with known nonrecent HIV infection from five African countries. AIDS Res Hum Retroviruses 28, 1177-1183 (2012).
14. R. Kassanjee et al., Independent assessment of candidate HIV incidence assays on specimens in the CEPHIA repository. AIDS 28, 2439-2449 (2014).
15. R. Brookmeyer, O. Laeyendecker, D. Donnell, S. H. Eshleman, Cross-sectional HIV incidence estimation in HIV prevention research. J Acquir Immune Defic Syndr 63 Suppl 2, S233-239 (2013).
16. J. E. Justman, O. Mugurungi, W. M. El-Sadr, HIV Population Surveys—Bringing Precision to the Global Response. N Engl J Med 378, 1859-1861 (2018).
17. T. J. Coates et al., Effect of community-based voluntary counselling and testing on HIV incidence and social and behavioural outcomes (NIMH Project Accept; HPTN 043): a cluster-randomised trial. Lancet Glob Health 2, e267-277 (2014).
18. C. S. Morrison et al., Hormonal contraceptive use and HIV disease progression among women in Uganda and Zimbabwe. J Acquir Immune Defic Syndr 57, 157-164 (2011).
19. C. S. Morrison et al., Plasma and cervical viral loads among Ugandan and Zimbabwean women during acute and early HIV-1 infection. AIDS 24, 573-582 (2010).
20. A. F. Longosz et al., Comparison of antibody responses to HIV infection in Ugandan women infected with HIV subtypes A and D. AIDS Res Hum Retroviruses 31, 421-427 (2015).
21. A. F. Longosz et al., Immune Responses in Ugandan Women Infected With Subtypes A and D HIV Using the BED Capture Immunoassay and an Antibody Avidity Assay. Jaids-J Acq Imm Def 65, 390-396 (2014).
22. D. Mohan et al., PhIP-Seq characterization of serum antibodies using oligonucleotide encoded peptidomes. Nature Protocols In Press, (2018).
23. B. Langmead, C. Trapnell, M. Pop, S. L. Salzberg, Ultrafast and memory-efficient alignment of short DNA sequences to the human genome. Genome Biol 10, R25 (2009).
24. T. Yuan et al., Improved analysis of phage immunoprecipitation sequencing (PhIP-Seq) data using a z-score algorithm. bioRxiv, (2018).
25. D. Monaco et al., Deconvoluting virome-wide antiviral antibody profiling data. bioRxiv, (2018).
26. S. K. Sharma, M. Soneja, HIV & immune reconstitution inflammatory syndrome (IRIS).

Indian J Med Res 134, 866-877 (2011).
27. G. Touloumi et al., Impact of HIV-1 subtype on CD4 count at HIV seroconversion, rate of decline, and viral load set point in European seroconverter cohorts. Clin Infect Dis 56, 888-897 (2013).
28. O. T. Ng et al., HIV type 1 polymerase gene polymorphisms are associated with phenotypic differences in replication capacity and disease progression. J Infect Dis 209, 66-73 (2014).
29. J. M. Baeten et al., HIV-1 subtype D infection is associated with faster disease progression than subtype A in spite of similar plasma HIV-1 loads. J Infect Dis 195, 1177-1180 (2007).
30. J. B. Margolick et al., Impact of inversion of the CD4/CD8 ratio on the natural history of HIV-1 infection. J Acquir Immune Defic Syndr 42, 620-626 (2006).
31. G. Pantaleo et al., The qualitative nature of the primary immune response to HIV infection is a prognosticator of disease progression independent of the initial level of plasma viremia. Proc Natl Acad Sci USA 94, 254-258 (1997).
32. J. L. Fahey et al., The prognostic value of cellular and serologic markers in infection with human immunodeficiency virus type 1. N Engl J Med 322, 166-172 (1990).
33. C. Costello et al., HLA-B*5703 independently associated with slower HIV-1 disease progression in Rwandan women. AIDS 13, 1990-1991 (1999).
34. Y. Huang et al., The role of a mutant CCR5 allele in HIV-1 transmission and disease progression. Nat Med 2, 1240-1243 (1996).
35. S. K. Wendel et al., Short communication: The impact of viral suppression and viral breakthrough on Limited-Antigen Avidity assay results in individuals with clade B HIV infection. AIDS Res Hum Retroviruses 33, 325-327 (2017).
36. X. Wei et al., Development of two avidity-based assays to detect recent HIV type 1 seroconversion using a multisubtype gp41 recombinant protein. AIDS Res Hum Retroviruses 26, 61-71 (2010).
37. J. Konikoff et al., Performance of a limiting-antigen avidity enzyme immunoassay for cross-sectional estimation of HIV incidence in the United States. PLoS One 8, e82772 (2013).
38. O. Laeyendecker et al., Identification and validation of a multi-assay algorithm for cross-sectional HIV incidence estimation in populations with subtype C infection. J Int AIDS Soc 21, (2018).
39. C. Kadelka et al., Distinct, IgG1-driven antibody response landscapes demarcate individuals with broadly HIV-1 neutralizing activity. J Exp Med 215, 1589-1608 (2018).

1. A method of identifying the cross-sectional incidence or duration of infection for a virus comprising the steps of:
obtaining a biological sample comprising antibodies from a subject who has one or more viral infections;
mixing the biological sample with a plurality of epitopes or peptides of the proteins from one or more viruses responsible for the one or more viral infections;
quantifying the amount of antibody binding to the plurality of epitopes or peptides of the proteins from the one or more viruses; and
estimating the cross-sectional incidence or duration of infection for the one or more viruses.

2. The method of claim 1 wherein the epitopes or peptides of the one or more virus responsible for the one or more viral infections are derived from, expressed in, or identified using a phage immunoprecipitation sequencing system (PhIP-Seq).

3. The method of claim 1 wherein the epitopes or peptides of the one or more virus responsible for the one or more viral infections are derived from, expressed in, or identified using a VirScan assay.

4. The method of claim 1 wherein the plurality of epitopes or peptides are modified by site-directed mutagenesis using alanine substitution or another method to alter the amino acid sequence of the peptides.

5. The method of claim 1 wherein the one or more viruses is HIV.

6. The method of claim 5 wherein the proteins are HIV proteins selected from the group comprising gp41, gp120, gag, and pol.

7. The method of claim 5 wherein the plurality of epitopes or peptides are selected from the group consisting of SEQ ID:1 to SEQ ID:309.

8. The method of claim 5 wherein the plurality of epitopes or peptides are selected from the group consisting of SEQ ID:1 to SEQ ID:309 in the range of two to twenty epitopes or peptides.

9. The method of claim 5 wherein the one or more epitopes or peptides are selected from the group consisting of SEQ ID: 1 to SEQ ID: 309 in the range of between ten to one hundred epitopes or peptides.

10. The method of claim 5 wherein the epitopes or peptides comprise SEQ ID:3, SEQ ID:22, SEQ ID:159 and SEQ ID:180.

11. The method of claim 2 wherein the one or more viral infections is HIV subtype C.

12. The method of claim 2 wherein the one or more viral infections is HIV subtype D.

13. The method of claim 12 wherein the virus is selected from the group consisting of HIV, EBV, other viruses, or a combination thereof.

14. The method of claim 1 wherein the epitopes or peptides are synthesized chemically.

15. The method of claim 14 wherein the eptiopes or peptides are used in a assay system that detects and/or quantify the binding of antibodies to one or more epitopes or peptides, either individually or in a multiplex (multi-assay) format.

16. The method of claim 15 wherein the assay system is selected from the group comprising an enzyme immunoassay, chemiluminescent assay, microparticle bead assay, electrochemiluminescent assay and a combination thereof.
